(19) United States
(12) Patent Application Publication
(10) Pub. No.: US 2022/0288119 A1
(43) Pub. Date: Sep. 15, 2022

(54) THIRD PARTY VIRUS-SPECIFIC T CELL COMPOSITIONS, AND METHODS OF MAKING AND USING THE SAME IN ANTI-VIRAL PROPHYLAXIS

(71) Applicant: Baylor College of Medicine, Houston, TX (US)

(72) Inventors: Juan Fernando Vera, Valdes, Houston, TX (US); Ann Marie Leen, Houston, TX (US); Ifigeneia Tzannou, Houston, TX (US)

(21) Appl. No.: 17/635,197

(22) PCT Filed: Aug. 14, 2020
(86) PCT No.: PCT/US20/46389

§ 371 Date: Feb. 14, 2022

Related U.S. Application Data
(60) Provisional application No. 62/887,806, filed on Aug. 16, 2019.

(51) Int. Cl. A61K 35/17; A61P 31/20; C12N 5/0783

(52) U.S. Cl. CPC A61K 35/17; A61P 31/20; C12N 5/0636; C12N 2710/16134

(57) ABSTRACT

The present disclosure includes compositions and methods for preventing viral infection and/or preventing reactivation of a latent virus in a subject. The methods involve prophylactically administering at least one antigen-specific T cell line from a third party donor and/or a donor minibank and/or a donor bank to a subject. The subject may be a patient who has received a transplant (e.g., a tissue, solid organ, or bone marrow transplant) or who is in need of such a transplant, or is immunosuppressed or in need of immunosuppressive therapy.

### CROSS-REFERENCE TO RELATED APPLICATIONS

This application claims priority to U.S. Provisional Application No. 62/887,806 filed Aug. 16, 2019, which is incorporated by reference herein in its entirety.

### FIELD

Embodiments of the disclosure concern at least the fields of cell biology, molecular biology, immunology, and medicine.

### BACKGROUND

Viral infections are a serious cause of morbidity and mortality after allogeneic hematopoietic stem cell transplantation (allo-HSCT), which is the treatment of choice for a variety of disorders. Post-transplant, however, graft versus host disease (GVHD), primary disease relapse and viral infections remain major causes of morbidity and mortality. Infections associated with viral pathogens include, but are not limited to CMV, BK virus (BKV), and adenovirus (AdV). Viral infections are detected in the majority of allograft recipients. Although available for some viruses, antiviral drugs are not always effective, highlighting the need for novel therapies. One strategy to treat these viral infections is with adoptive immunotherapy, e.g., adoptive T cell transfer.

Adoptive immunotherapy involves implanting or infusing disease-specific and/or engineered cells such as T cells, (e.g., antigen-specific T cells or chimeric antigen receptor (CAR)-expressing T cells), into individuals with the aim of recognizing, targeting, and destroying disease-associated cells. Adoptive immunotherapies have become a promising approach for the treatment of many diseases and disorders, including cancer, post-transplant lymphoproliferative disorders, infectious diseases (e.g., viral and other pathogenic infections), and autoimmune diseases.

There are two primary types of adoptive immunotherapies. Autologous immunotherapy involves isolation, production, and/or expansion of cells such as T cells, (e.g., antigen-specific T cells) from the patient and storage of the patient-harvested cells for re-administration into that same patient as needed. Allogeneic immunotherapy involves two individuals: the patient and a healthy donor. Cells, such as T cells (e.g., antigen-specific T cells), are isolated from the healthy donor and then produced, and/or expanded and banked for administration to a patient with a matching (or partially matching) human leukocyte antigen (HLA) type based on a number of HLA alleles. HLA is also called the Human major histocompatibility complex (MHC). With this approach, one can extract cells from the donor of the stem cells, expand virus-specific populations ex vivo and, finally, infuse the T cell product into the stem cell transplant recipient to treat the viral infection in the recipient. For example, in vitro expanded donor-derived virus-specific T cells targeting Adv, EBV, CMV, BK, HHV6 have shown to be safe and effective when adoptively transferred to stem cell transplant patients with viral infections (Gerdemann et al., 2012). Third party donor-derived virus-specific T cells targeting such viruses have also been shown to be safe, but are only considered suitable to treat ongoing viral infections. This is because third party virus-specific T cells and other cell therapies that are generated from third party cells are recognized as non-self by the recipient immune system and are expected to be rejected.

Viral infections such as Adv, EBV, CMV, BK, HHV6, HSV-1, HSV-2, HHV8, HBV, influenza, parainfluenza, HMPV, VZV, and others are also concerns for patients who are immunocompromised for reasons other than transplantation therapy, such as age (young age or old age), immune deficiency, or treatment with immunosuppressive therapies for certain cancers or autoimmune diseases. There is a need in the art for therapies that better control or prevent the various causes of morbidity and mortality that occur in immunocompromised patients due to viral infection. This disclosure addresses this and other needs.

### SUMMARY

The present disclosure includes methods for preventing or controlling a viral infection or the reactivation of a latent virus via prophylactic administration of a third-party allogeneic T cell therapy. In embodiments, the method comprises prophylactically administering to a patient a first antigen-specific T cell line that is a polyclonal third party T cell line, said T cell line comprising antigen specificity for one or more viral antigen. In embodiments, the T cell line comprises an HLA type that matches the patient's HLA type on 2 or more HLA alleles. In embodiments, the prophylactic administration is such that the patient does not show evidence of an active viral infection or of reactivation of the latent virus when the T cell line is administered. For example, in embodiments, the patient is administered a polyclonal third party T cell line with T cells specific for one or more viruses, wherein the patient does not have an active infection with respect to the one or more viruses, or wherein the patient does not have any active viral infection. In embodiments, the patient has no detectable viremia or viruria when the T cell line is administered.

In embodiments, the patient is at a higher risk than an average person in the general population of contracting a viral infection or of having a latent virus reactivate. For example, in embodiments, viral infection poses a greater risk to the patient's health or life than such an infection would pose to an average person in the general population. In embodiments, the patient has an absolute lymphocyte count of less than about 1200, less than about 1000, less than about 900, less than about 800, less than about 700, less than about 600, or less than about 500 lymphocytes per μL blood. In embodiments, the patient lacks endogenous T cells. In embodiments, the patient is seropositive for any one or more of AdV, BKV, CMV, EBV, HHV6, HHV8, RSV, influenza, parainfluenza (PIV), human metapneumovirus (hMPV), SARS-CoV-2 and HBV.

In embodiments, the patient is immunocompromised. In embodiments, the patient is immunocompromised due to a disease or condition, due to a treatment the patient received to treat a disease or condition, or due to age. In embodiments, the patient is scheduled to undergo or has undergone a hematopoietic stem cell transplant (HSCT), solid organ transplant, or tissue transplant. In embodiments, the subject is in need of HSCT therapy, a solid organ transplant, or a tissue transplant. For example, in embodiments, the patient is in need of or has had a kidney, liver, heart, heart valve, lung, pancreas, intestine, cornea, musculoskeletal, connective tissue, skin, hand, or face transplant. In embodiments, the patient is receiving immunosuppressive therapy to prevent rejection of the transplant. In embodiments, the subject has cancer, e.g., a leukemia, myeloma, or lymphoma. In embodiments, the subject has a cancer and is in need of HSCT. In embodiments, the subject has one or more nonmalignant diseases and is in need of HSCT. For example, in embodiments, the subject has aplastic anemia, a myelodysplastic syndrome, or an immunodeficiency syndrome. In embodiments, the subject is receiving immunosuppressive or chemotherapeutic therapy as a cancer treatment.

In embodiments, the treatment the patient received to treat a disease or condition is selected from the group consisting of reduced intensity conditioning, myeloablative conditioning, non-myeloablative conditioning, chemotherapy, and immunosuppressive drugs.

In embodiments, the patient is immunocompromised due to age, e.g., due to young or old age. In embodiments, the patient is less than 1 year of age, less than 9 months of age, less than 6 months of age, less than 3 months of age, or less than 1 month of age. In embodiments, the patient is more than 65 years of age, more than 70 years of age, more than 75 years of age, or more than 80 years of age.

In embodiments, the patient has an immune deficiency condition. For example, in embodiments, the subject has a primary immune deficiency, e.g., a primary immune deficiency disease (PIDD). In embodiments, the patient has an acquired immune deficiency condition. In embodiments, the subject has a human immunodeficiency virus (HIV) infection, and/or the subject has acquired immune deficiency syndrome (AIDS).

In embodiments, the methods provided herein for preventing or controlling a viral infection or the reactivation of a latent virus via prophylactic administration of a third-party allogeneic T cell therapy comprise prophylactically administering a first polyclonal third party antigen-specific T cell, wherein the T cell line is administered to the patient a plurality of times (e.g., 2, 3, 4, 5, 6, or more times). For example, in embodiments, the first antigen-specific T cell line is administered to the patient in a second administration 4-12 weeks after a first administration. In embodiments, the first antigen-specific T cell line is administered to the patient in a second administration about 4-12 weeks after a first administration. In embodiments, the first antigen-specific T cell line is administered to the patient every 4-12 weeks, e.g., every 4 weeks, every 5 weeks, every 6 weeks, every 7 weeks, every 8 weeks, every 9 weeks, every 10 weeks, every 11 weeks, or every 12 weeks. In embodiments, the first antigen-specific T cell line is administered to the patient about every 4-12 weeks, e.g., about every 4 weeks, about every 5 weeks, about every 6 weeks, about every 7 weeks, about every 8 weeks, about every 9 weeks, about every 10 weeks, about every 11 weeks, or about every 12 weeks. In embodiments, the time between administrations of the first antigen-specific T cell line varies. For example, in embodiments, after the first administration of the first antigen-specific T cell line, the patient is monitored for the persistence of the T cell line and/or is monitored for viremia and/or viruria, and the first antigen-specific T cell line is administered in subsequent administrations accordingly. In embodiments, the first antigen-specific T cell line is administered to the subject repeatedly for the duration of time that the subject is at risk and/or at high risk of a viral infection or reactivation of latent virus, and/or until the patient is no longer immunocompromised.

In embodiments, as an alternative to or in addition to multiple administrations of the first antigen-specific T cell line, the patient is administered a composition comprising a peptide or whole antigen that corresponds to an antigen for which the first antigen-specific T cell line is specific. In embodiments, the composition is administered after the first antigen-specific T cell line. In embodiments, the composition comprising the peptide or whole antigen is administered to the subject 4 to 12 weeks after administration of the first antigen-specific T cell line. In embodiments, the composition comprising the peptide or whole antigen is administered to the subject about 4 to about 12 weeks after administration of the first antigen-specific T cell line. In embodiments, the composition is administered multiple times after the first antigen-specific T cell line. For example, in embodiments, the composition comprising the peptide or whole antigen is administered to the subject every 4 weeks, every 5 weeks, every 6 weeks, every 7 weeks, every 8 weeks, every 9 weeks, every 10 weeks, every 11 weeks, or every 12 weeks after administration of the first antigen-specific T cell line. In embodiments, the composition comprising the peptide or whole antigen is administered to the subject about every 4 weeks, about every 5 weeks, about every 6 weeks, about every 7 weeks, about every 8 weeks, about every 9 weeks, about every 10 weeks, about every 11 weeks, or about every 12 weeks after administration of the first antigen-specific T cell line. In embodiments, the composition comprising the peptide or whole antigen is administered 2, 3, 4, 5, 6, or more times. In embodiments, the composition comprising the peptide or whole antigen is administered after the first antigen-specific T cell line is administered to the subject, and is repeatedly administered for the duration of time that the subject is at risk and/or at high risk of a viral infection or reactivation of latent virus, and/or until the patient is no longer immunocompromised. In embodiments, the composition comprising the peptide or whole antigen further comprises an adjuvant.

In embodiments, the methods provided herein further comprise administering to the patient one or more second antigen-specific T cell lines; or administering to the patient 2, 3, 4, 5, 6, 7, 8, 9, or 10 more second antigen-specific T cell lines. In embodiments, the first and the second antigen-specific T cell lines are administered to the patient concurrently or sequentially. In embodiments, the one or more second antigen-specific T cell lines are administered to the patient a plurality of times. For example, in embodiments, the one or more second antigen-specific T cell lines are administered to the patient every 4-12 weeks, e.g., every 4 weeks, every 5 weeks, every 6 weeks, every 7 weeks, every 8 weeks, every 9 weeks, every 10 weeks, every 11 weeks, or every 12 weeks. In embodiments, the one or more second antigen-specific T cell lines are administered to the patient about every 4-12 weeks, e.g., about every 4 weeks, about every 5 weeks, about every 6 weeks, about every 7 weeks, about every 8 weeks, about every 9 weeks, about every 10 weeks, about every 11 weeks, or about every 12 weeks. In embodiments, the one or more second antigen-specific T cell line is administered until the patient is no longer immunocompromised. In embodiments, the second antigen-specific T cell line comprises the same antigen specificity as the first antigen-specific T cell line, but is generated from a different donor. In embodiments, the second antigen-specific T cell line comprises some of the same antigen specificity as the first antigen specific T cell line. In embodiments, the second antigen-specific T cell line comprises different antigen specificity than the first antigen-specific T cell line. In embodiments, the 2 or more HLA alleles that are matched between the patient and the first antigen-specific T cell line and/or any second antigen-specific T cell line comprises at least 2 HLA Class I alleles; at least 2 HLA Class II alleles; or at least 1 HLA Class I allele and at least 1 HLA Class II allele. In embodiments, the HLA types are HLA-A, HLA-B, HLA-DR, and/or HLA-DQ.

In embodiments, the third party VSTs have not been genetically modified. In embodiments, the third party VSTs have not been modified to reduce recognition and rejection by host immune cells. For example, in embodiments, the third party VSTs have not been modified to remove HLA and/or TCR molecules from the VST cell surface.

In embodiments, the present disclosure provides methods or preventing or controlling a viral infection or the reactivation of a latent virus via prophylactic administration of a third-party allogeneic T cell therapy comprising prophylactically administering a first polyclonal third party antigen-specific T cell, wherein the viral infection is from a virus selected from EBV, CMV, Adenovirus, BK, JC virus, HHV6, RSV, Influenza, Parainfluenza, Bocavirus, Coronavirus (e.g., SARS-CoV-2), LCMV, Mumps, Measles, human Metapneumovirus, Parvovirus B, Rotavirus, merkel cell virus, herpes simplex virus, HPV, HIV, HTLV1, HHV8 and West Nile Virus, zika virus, ebola. In embodiments, the first and/or second antigen-specific T cell line comprises antigen specificity for at least one antigen or a portion thereof from a single virus. In embodiments, the single virus is selected from EBV, CMV, Adenovirus, BK, JC virus, HHV6, RSV, Influenza, Parainfluenza, Bocavirus, Coronavirus, LCMV, Mumps, Measles, human Metapneumovirus, Parvovirus B, Rotavirus, merkel cell virus, herpes simplex virus, HPV, HIV, HTLV1, HHV8 and West Nile Virus, zika virus, ebola. In particular embodiments, the single virus is HBV or HHV8. In embodiments, the first antigen-specific T cell line comprises specificity for two or more antigens or a portion thereof from the single virus.

In embodiments, the present disclosure provides methods or preventing or controlling a viral infection or the reactivation of a latent virus via prophylactic administration of a third-party allogeneic T cell therapy comprising prophylactically administering a first polyclonal third party antigen-specific T cell, wherein the first antigen-specific T cell line comprises antigen specificity for at least one antigen or a portion thereof, from 1-10 different viruses. In embodiments, the first antigen-specific T cell line comprises antigen specificity for 2-5 antigens from each of at least two different viruses or at least a portion of 2-5 antigens from each of at least two different viruses. In embodiments, the second antigen-specific T cell line comprises antigen specificity for at least one antigen or a portion thereof, from 1-10 different viruses. In embodiments, the second antigen-specific T cell line comprises antigen specificity for 2-5 antigens from each of at least two different viruses or at least a portion of 2-5 antigens from each of at least two different viruses.

In embodiments, the antigen is a viral antigen from a virus selected from EBV, CMV, Adenovirus, BK, JC virus, HHV6, RSV, Influenza, Parainfluenza, Bocavirus, Coronavirus, LCMV, Mumps, Measles, human Metapneumovirus (HMPV), Parvovirus B, Rotavirus, merkel cell virus, herpes simplex virus, HPV, HIV, HTLV1, HHV8, West Nile Virus, zika virus, and ebola. In embodiments, the first and/or the second antigen-specific T cell comprises specificity for at least one antigen from each of the following viruses: RSV, Influenza, Parainfluenza, and HMPV. In embodiments, the Influenza antigens are selected from influenza A antigens NP1, MP1, and a combination thereof; the RSV antigens are selected from N, F, and a combination thereof; the hMPV antigens are selected from F, N, M2-1, M, and a combination thereof; and the PIV antigens are selected from M, HN, N, F, and a combination thereof.

In embodiments, the first and/or the second antigen-specific T cell comprises specificity for at least one antigen from each of the following viruses: EBV, CMV, adenovirus, BK, HHV6. In embodiments, EBV antigens are selected from LMP2, EBNA1, BZLF1, and a combination thereof; the CMV antigens are selected from IE1, pp65, and a combination thereof; the adenovirus antigens are selected from Hexon, Penton, and a combination thereof; the BK virus antigens are selected from VP1, large T, and a combination thereof; and the HHV6 antigens are selected from U90, U11, U14, and a combination thereof.

In embodiments, the first and/or the second antigen-specific T cell comprises specificity for at least one antigen from HBV. In embodiments, the antigens from HHV8 are selected from LANA-1 (ORF3); LANA-2 (vIRF3, K10.5); vCYC (ORF72); RTA (ORF50); vFLIP (ORF71); Kaposin (ORF12, K12); gB (ORF8); MIR1 (K3); SSB (ORF6); TS(ORF70), and a combination thereof.

In embodiments, first and/or the second antigen-specific T cell comprises specificity for at least one antigen from HHV8. In embodiments, the antigens from HBV are selected from HBV core antigen, HBV Surface Antigen, and a combination of HBV core antigen and HBV Surface Antigen.

In embodiments, the antigen-specific T cells provided herein for use in the methods provided herein are produced by culturing, in the presence of the antigens or a portion thereof, mononuclear cells from a suitable donor having an HLA type that matches the patient's HLA type on 2 or more HLA alleles. In embodiments, the antigen-specific T cells are produced by culturing, in the presence of pepmixes spanning the antigens, or a portion thereof, mononuclear cells from a suitable donor having an HLA type that matches the patient's HLA type on 2 or more HLA alleles. In embodiments, the culturing is in the presence of IL4 and IL7. In embodiments, the pepmix comprises 15 mer peptides. In embodiments, the peptides in the pepmix that span the antigen overlap in sequence by 11 amino acids. In embodiments, the antigen-specific T cells provided herein for use in the methods provided herein are produced by culturing, in the presence of the antigens or a portion thereof, peripheral blood mononuclear cells (PBMCs) from a suitable donor having an HLA type that matches the patient's HLA type on 2 or more HLA alleles. In embodiments, the antigen-specific T cells are produced by culturing, in the presence of pepmixes spanning the antigens, or a portion thereof, PBMCs from a suitable donor having an HLA type that matches the patient's HLA type on 2 or more HLA alleles. In embodiments, the culturing is in the presence of IL4 and IL7. In embodiments, the pepmix comprises 15 mer peptides. In embodiments, the peptides in the pepmix that span the antigen overlap in sequence by 11 amino acids. In embodiments, the present disclosure provides methods or preventing or controlling a viral infection or the reactivation of a latent virus via prophylactic administration of a third-party allogeneic T cell therapy comprising prophylactically administering a first polyclonal third party antigen-specific T cell and optionally one or more second polyclonal third party antigen-specific T cell line, wherein the first and/or one or more of each second T cell lines persist in vivo for at least about 4 weeks, at least about 6 weeks, at least about 8 weeks, at least about 10 weeks, or at least about 12 weeks. In embodiments, the first and/or one or more of each second T cell lines persist in vivo for at least about 4 weeks, at least about 6 weeks, at least about 8 weeks, at least about 10 weeks, or at least about 12 weeks absent any active infection in the patient. For example, in some embodiments, the first and/or one or more of each second T cell lines persist in vivo for 4 weeks, 6 weeks, 8 weeks, 10 weeks, 12 weeks, or more than 12 weeks. In embodiments, the first and/or one or more of each second T cell lines persist in vivo for 4 weeks, 6 weeks, 8 weeks 10 weeks, 12 weeks, or more than 12 weeks absent any active infection in the patient

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. is a schematic showing general manufacturing concepts of the antigen-specific T cell lines.

FIG. 2 is a flowchart of manufacturing of the antigen-specific T cell lines.

FIG. 3A-3D. Characteristics of generated CMVST lines and degree of matching with screened subjects (3A) T cell expansion of CMVSTs achieved over a 20-day period based on cell counting using trypan blue exclusion. (n=8). (3B) Phenotype of the expanded CMVST lines on the day of cryopreservation (mean±SEM, n=8) and (3C) frequency of antigen-specific T cells as determined by IFN-γ ELISpot assay after overnight stimulation of CMVSTs with IE1 and pp65 antigen-spanning pepmixes. Results are reported as spot forming cells (SFC) per 2×10⁵ VSTs plated. CMVST lines with a total of 2:30 SFC/2×10⁵ were considered to be positive. (n=8). (3D) Number of matching HLA antigens (of 8 total) of CMVST lines identified for clinical use with recipient HLA of screened patients (n=29).

FIG. 4. Treatment outcomes in individual patients infected with cytomegalovirus (CMV). Depiction of plasma CMV viral loads (IU/mL) in patients 2 weeks prior to (viral load level closest to week −2), immediately before (pre) and after (post) infusion (weeks 2, 4 and 6) of CMVSTs. Arrows indicate infusion timepoints.

FIG. 5A-5B. Frequency of CMV specific T cells in vivo. (5A) Frequency of CMVSTs in the peripheral blood before (pre) and after (post) infusion, as measured by IFN-γ ELISpot assay after overnight stimulation with IE1 and pp65 viral pepmixes. Results are expressed as spot-forming cells (SFCs) per 5×10⁵ input cells (mean±SEM, n=10). (5B) Persistence of infused CMVSTs in individual patients. Frequency of T cells in peripheral blood as measured by IFN-γ ELISpot assay after stimulation with epitope-specific CMV peptides with restriction to HLA antigens exclusive to the CMVST line or shared between the recipient and the CMVST line.

FIG. 6 shows the relative presence of immune responses against peptides presented in the context of HLA-A2 (CMV-specific), DR13 (3^{rd} party VST only) and DR3 (patient only) at 2 weeks and 4 weeks after VST infusion.

FIG. 7 shows the decrease in BKV urine viral load (dotted line) corresponding with BK-specific T cell expansion (bars) after infusion of VSTs to treat the patient's BKV infection.

FIG. 8 shows the reactivation of CMV (dotted line; urine viral load) at 2 weeks after VST infusion, expansion of CMV-specific 3^{rd} party VSTs (bars), and subsequent resolution of viral load by week 12.

FIG. 9A-9E show the detection of third party VSTs persisting in patients treated for other viruses. FIG. 9A shows that in a patient treated for BK, EBV and/or HHV6-specific cells were detectable for at least 3 weeks after VST infusion. FIG. 9B shows that in a 2^{nd} patient treated for BK, CMV-specific T cells expanded after week 1 and persisted for at least 4 weeks after VST infusion. FIG. 9C shows that in a patient treated for AdV, CMV specific T cells were detectable for at least 3 weeks after VST infusion. FIG. 9D shows that for another patient treated for AdV, CMV-specific T cells expanded after week 2 and were detectable at least 4 weeks after VST infusion. FIG. 9E shows that in a patient treated for BK, CMV-specific T cells expanded and were detectable for at least 6 weeks after VST infusion.

FIG. 10 is a schematic depiction of the prophylactic protection mediated by VST T cells in immunocompromised individuals.

### DETAILED DESCRIPTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

HSCT is a potentially curative therapy for life-threatening hematopoietic malignancies, including acute leukemia, as well as nonmalignant diseases including aplastic anemia, myelodysplastic syndromes and immunodeficiency syndromes. However, the preparative regimens associated with HSCT result in profound deficiencies in the cellular as well as humoral components of the immune system leaving the patients vulnerable to viral infections. The risk for infection and the spectrum of infectious syndromes differs by type of transplant (higher risk for allogeneic transplant); type of allogeneic donor (higher risk with unrelated or mismatched donor); type of conditioning regimen (higher risk with intensive myeloablative regimen); type of stem cell graft (higher risk with cord blood); type of graft manipulation (higher risk with T cell depletion) and use of immunosuppressive drugs like antithymocyte globulin (ATG). Nevertheless, viral complications remain one of the leading causes of morbidity and nonrelapse mortality in allogeneic HSCT (allogeneic-HSCT) recipients.

Antiviral prophylaxes in HSCT recipients are sparse, toxic and fail to address the underlying deficiency—namely the lack of endogenous immunity—thus any conferred benefit tends to be temporary leaving patients at risk for recurrence. Cutler et al. 2005. Therefore, there is an unmet need for novel prophylactic strategies that are safe and efficacious. Adoptive transfer of stem cell donor-derived VSTs has been used in attempts to provide prophylactic therapy against infection in allogeneic-HSCT recipients. However, it is well understood in the field that with respect to third party allogeneic VSTs (i.e., VSTs derived from third party donors rather than the stem cell donor), only methods for treating active viral infections, and not prophylactic methods, are feasible since third party cells are expected to be rapidly rejected and fail to persist in the recipient.

The present inventors made the surprising discovery that third party allogeneic VSTs persist, and retain the ability to expand, in the recipient in the absence of an active viral infection for which the VSTs have specificity. In fact, the third party allogeneic VSTs are capable of persisting for several weeks and then expanding immediately upon infection with or reactivation of the virus for which they are specific. Thus, the present disclosure provides an unexpected and highly efficient method for preventing or controlling a viral infection or the reactivation of a latent virus via a third-party allogeneic T cell therapy. In particular, the methods and compositions provided herein provide an immediately available, safe, and effective protection against dangerous viral infections in patients at high risk. Such patient populations include recipients of allogeneic-HSCT as well as patients who are immunocompromised and at high risk of dangerous viral infections for other reasons.

### General Methods

The practice of the present invention will employ, unless otherwise indicated, conventional techniques of cell culturing, molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” By way of example, “an element” means one element or more than one element. Some embodiments of the invention may consist of or consist essentially of one or more elements, method steps, and/or methods of the invention. It is contemplated that any method or composition described herein can be implemented with respect to any other method or composition described herein.

The term “about” when immediately preceding a numerical value means ±0% to 10% of the numerical value, ±0% to 10%, ±0% to 9%, ±0% to 8%, ±0% to 7%, ±0% to 6%, ±0% to 5%, ±0% to 4%, ±0% to 3%, ±0% to 2%, ±0% to 1%, ±0% to less than 1%, or any other value or range of values therein. For example, “about 40” means ±0% to 10% of 40 (i.e., from 36 to 44).

The term “and/or” is used in this disclosure to mean either “and” or “or” unless indicated otherwise.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “disorder” is used in this disclosure to mean, and is used interchangeably with, the terms disease, condition, or illness, unless otherwise indicated.

An “effective amount” when used in connection with a therapeutic agent (e.g., an antigen specific T cell product or cell line disclosed herein) is an amount effective for treating or preventing a disease or disorder in a subject as described herein.

The term “e.g.” is used herein to mean “for example,” and will be understood to imply the inclusion of a stated step or element or group of steps or elements but not the exclusion of any other step or element or group of steps or elements.

By “optional” or “optionally,” it is meant that the subsequently described event or circumstance may or may not occur, and that the description includes instances where the event or circumstance occurs and instances in which it does not.

The term “viral antigen” as used herein refers to an antigen that is protein in nature and is closely associated with the virus particle. In specific embodiments, a viral antigen is a coat protein.

Specific examples of viral antigen include at least antigens from a virus selected from Epstein Barr Virus (EBV), Cytomegalovirus (CMV), Adenovirus (AdV), BK virus (BKV), JC virus (JCV), Human Herpes Virus 6 (HHV6), Respiratory Syncytial Virus (RSV), Influenza, Parainfluenza, Bocavirus, Coronavirus, Lymphocytic Choriomeningitis Virus (LCMV), Mumps, Measles, human Metapneumovirus (HMPV), Parvovirus B, Rotavirus, Merkel cell virus, herpes simplex virus (HSV), Human Papilloma Virus (HPV), Hepatitis B Virus (HBV), Human Immunodeficiency Virus (HIV), Human T Cell Leukemia Virus type 1 (HTLV1), Human Herpes Virus 8 (HHV8), West Nile Virus, Zika Virus, and Ebola Virus.

The term “virus-specific T cells” or “VSTs” or “virus-specific T cell lines” or “VST cell lines” are used interchangeably herein to refer to T cell lines, e.g., as described herein, that have been expanded and/or manufactured outside of a subject and that have specificity and potency against a virus or viruses of interest. The VSTs provided herein are third party VSTs. The VSTs may be monoclonal or oligoclonal, in embodiments. In particular embodiments the VSTs are polyclonal. As described herein, in embodiments, a viral antigen or several viral antigens are presented to native T cells or memory T cells in peripheral blood mononuclear cells and the native CD4+ and/or CD8+ T cell populations with specificity for the viral antigens(s) expand in response. For example, a virus-specific T cell for EBV in a sample of PBMCs obtained from a suitable donor can recognize (bind to) an EBV antigen (e.g., a peptidic epitope from an EBV antigen, optionally presented by an MHC) and this can trigger expansion of T cells specific for EBV. In another example, a virus-specific T cell for BK virus in a sample of PBMCs obtained from a suitable donor a virus-specific T cell for adenovirus in the sample of PBMCs can respectively recognize and bind to a BK virus antigen and an adenovirus antigen (e.g., a peptidic epitope from a BK virus antigen and an adenovirus antigen, respectively, optionally presented by an MHC) and this can trigger expansion of T cells specific for a BK virus and T cells specific for an adenovirus.

As used herein, the term “cell therapy product” refers to a cell line, e.g., as described herein, expanded and/or manufactured outside of a subject. For example, the term “cell therapy product” encompasses a cell line produced in a culture. The cell line may comprise or consist essentially of effector cells. The cell line may comprise or consist essentially of T cells. For example, the term “cell therapy product” encompasses an antigen specific T cell line produced in a culture. Such antigen specific T cell lines include in some instances expanded populations of memory T cells, and expanded populations of T cells produced by stimulating naïve T cells. In particular, the term “cell therapy product” in embodiments includes a virus specific T cell line. The cell line may be monoclonal or oligoclonal. In particular embodiments, the cell line is polyclonal. Such polyclonal cells lines comprise, in embodiments, a plurality of expanded populations of cells (e.g., antigen specific T cells) with divergent antigen specificity. For example, one non-limiting example of a cell line encompassed by the term “cell therapy product” comprises a polyclonal population of virus specific T cells comprising a plurality of expanded clonal populations of T cells, at least two of which respectively have specificity for different viral antigens. Such polyclonal virus specific T cells are known in the art and are disclosed in various patent applications filed by the inventors including WO2011028531, WO2013119947, WO2017049291, and PCT/US2020/024726, each of which is incorporated herein by reference in its entirety.

The term “donor minibank” as used herein refers to a cell bank comprising a plurality of cell therapy products (e.g., antigen-specific T cell lines) collectively derived from a diverse pool of donors such that the donor minibank contains at least one well-matched cell therapy product (e.g., antigen-specific T cell line) for a defined percentage of patients in a target patient population. For example, in certain embodiments, the donor minibanks described herein include at least one well-matched cell therapy product (e.g., antigen-specific T cell line) for at least 95% of a target patient population (such as, e.g., allogenic hematopoietic stem cell transplantation recipients or immunocompromised subjects). The term “donor bank” as used herein refers to a plurality of donor minibanks. In various embodiments, it is beneficial to create several non-redundant minibanks for inclusion in a “donor bank” to ensure the availability of two or more well-matched cell therapy products for each prospective patient. Cell banks may be cryopreserved. Cryopreservation methods are known in the art and may include, e.g., storage of the cell therapy products (e.g., antigen-specific T cell lines) at −70° C., e.g., in vapor-phase liquid nitrogen in a controlled-access area. Separate aliquots of cell therapy products may be prepared and stored in containers (e.g., vials) in multiple, validated, liquid nitrogen dewars. Containers (e.g., vials) may be labeled with unique identification numbers enabling retrieval.

As used herein, the terms “patient” or “subject” are used interchangeably to refer to any mammal, including humans, domestic and farm animals, and zoo, sports, and pet animals, such as dogs, horses, cats, cattle, sheep, pigs, goats, rats, guinea pigs, or non-human primates, such as a monkeys, chimpanzees, baboons or rhesus. One preferred mammal is a human, including adults, children, and the elderly.

As used herein, the term “potential donor” refers to an individual (e.g., a healthy individual) with seropositivity for the antigen or antigens that will be targeted by the cell therapy products (e.g., antigen specific T cells) disclosed herein. In embodiments, all potential donors eligible for inclusion in the donor pools are prescreened and/or deemed seropositive for the target antigen(s).

The term “target patient population” is used in embodiments herein to describe a plurality of patients (or “subjects” interchangeably) in need of a cell therapy product described herein (e.g., an antigen specific T cell product). In embodiments, this term encompasses the entire worldwide allogeneic HSCT population. In embodiments, this term encompasses the entire US allogeneic HSCT population. In embodiments, this term encompasses all patients included in the National Marrow Donor Program (NMDP) database, available at the worldwide web address bioinformatics.bethematchclinical.org. In embodiments, this term encompasses all patients included in the European Society for Blood and Marrow Transplantation (EBMT) database, available at the worldwide web address: ebmt.org/ebmt-patient-registry. In embodiments, this term encompasses the entire worldwide allogeneic HSCT population of children ages ≤16 years. In embodiments, this term encompasses the entire US allogeneic HSCT population of children ages ≤16 years. In embodiments, this term encompasses the entire worldwide allogeneic HSCT population of children ages ≤5 years. In embodiments, this term encompasses the entire US allogeneic HSCT population of children ages ≤5 years. In embodiments, this term encompasses the entire worldwide allogeneic HSCT population of individuals ages ≥65. In embodiments, this term encompasses the entire US allogeneic HSCT population of individuals ages ≥65.

The term “prevent” or “preventing” with regard to a subject refers to keeping a disease or disorder from afflicting the subject or to reducing the severity of a disease or disorder that would otherwise occur in the subject. Prophylactic treatment encompasses preventing. For instance, preventing can include administering to the subject a compound disclosed herein before a subject is afflicted with a disease, is infected with a virus, or undergoes reactivation of a latent virus infection. In embodiments, preventing means that the administration of the prophylactic treatment will keep the subject from being afflicted with the disease, keep the subject from being infected with the virus, or keep the latent virus from reactivating. Prophylactic treatment also encompasses controlling. For example, controlling a viral infection means that the administration of the prophylactic treatment is prior to the viral infection, wherein the prophylactic treatment controls and/or resolves the subsequent viral infection before it causes significant disease, morbidity or mortality. Controlling a viral infection also means that the administration of the prophylactic treatment is prior to reactivation of a latent virus, and will control and/or resolve the reactivated virus before it causes significant disease, morbidity or mortality. Accordingly, a method provided herein for “controlling” a viral infection means that the viral infection is prevented or readily cleared by a previously administered, prophylactic treatment with the third party VSTs provided herein.

The terms “treat”, “treating”, “treatment” and the like, as used herein, unless otherwise indicated, refers to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the disease, condition, or disorder. In some instances, treatment is curative or ameliorating.

Reference herein to the term “third party” means a subject (e.g., a patient) that is not the same as a donor. So, for example, reference to administering to a subject a “third party antigen-specific T cell product” (e.g., a third party VST product) means that the product is derived from donor tissue (e.g., PBMCs isolated from the donor's blood) and the subject (e.g., patient) is not the same subject as the donor. In embodiments, the third party antigen-specific T cell product is an “off the shelf” product in that it is prospectively generated and may be stored (e.g., cryopreserved) until use. Such products are immediately available for use in a subject in need thereof as opposed to autologous cell products or individualized donor cell products (i.e., cell products generated from cells from the same donor that donated the cells or tissue or organ to the subject, or a donor that is otherwise selected for a particular level of HLA matching). Thus, such products are advantageous since they can be administered without delay to patients in need of immediate therapy. In various embodiments, an allogeneic cell therapy (e.g., an allogeneic antigen-specific T cell therapy) is a “third party” cell therapy. The term “VST” as used herein means virus-specific T cell.

The terms “administering”, “administer”, “administration” and the like, as used herein, refer to any mode of transferring, delivering, introducing, or transporting a therapeutic agent to a subject in need of treatment with such an agent. Such modes include, but are not limited to, intraocular, oral, topical, intravenous, intraperitoneal, intramuscular, intradermal, intranasal, and subcutaneous administration.

In various embodiments, the term “well-matched” is used herein in reference to a given patient and a given cell therapy product (e.g., an antigen specific T cell line) to describe when the patient and the cell therapy product shares (i.e., is matched on) at least two HLA alleles.

Other objects, feature and advantages of the present invention will become apparent from the following detailed description. It should be understood, however, that the detailed description and specific examples, while indicating specific embodiments of the invention, are given by way of illustration only, since various changes and modifications within the spirit and scope of the invention will become apparent to those skilled in the art from this detailed description.

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

### Overview

It has long been understood that the immune system evolved for the purpose of recognizing and eliminating pathogens from the body. The immune system accomplishes this by distinguishing “self” from “non-self”. Immune cells react only against molecules, proteins, cells, or tissues that they recognize as non-self. Non-self encompasses any foreign material, including pathogens as well as biologic material that is not immunologically matched (HLA matched, as described above) with the immune cells. Therefore, cells or tissues from a non-immunologically matched donor, if infused into a body where they are recognized by immune cells as non-self, will be rejected (i.e., attacked, destroyed, and/or removed) by those immune cells.

Thus, it is expected that cell therapy products, e.g., third party VSTs, that are only partially matched with an immunocompromised stem cell transplant recipient will circulate only until a time that the donor's cells engraft and begin to repopulate the recipient, at which point the cell therapy product (e.g., VSTs) will be rejected by the patient's reconstituted immune system. Because of the expected rejection of third party VSTs, they have been used only for treating an active viral infection such as a new infection or an already reactivated latent virus infection. That is, when an infection or reactivation is detected in a patient, third party VSTs that are already expanded and specific for the infecting or reactivated virus can be infused for immediate response against the virus, a scenario in which rejection of the third party VSTs is not a concern. The expected rejection of third party cells before they can serve any protective purpose in any other host environment is well recognized in the field and indeed, other allogeneic off-the-shelf cell products (e.g., chimeric antigen receptor (CAR) T cells or anti-tumor T cell grafts) are typically modified to reduce recognition and rejection by host immune cells (Liu et al., Cell Research (2017); Kagoya et al. 2020). Surprisingly, the present inventors found that the third party VSTs provided herein could be administered to patients in a prophylactic method, and yet remain in the circulation for several weeks, even without any modification to reduce recognition by host immune cells. Moreover, the VSTs were capable of expansion upon infection with a virus or upon reactivation of a latent virus several weeks after administration. Thus, the present disclosure provides an off-the-shelf, third party VST product which provides both effective prevention or control of virus infections (including reactivated latent viruses), and the advantages of immediate availability, standardization, and availability for multiple re-dosing.

In embodiments, the VSTs circulate in the recipient for at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, or at least 18 weeks, inclusive of all ranges and subranges therebetween. In one embodiment, the VSTs circulate in the recipient for at least 12 weeks.

The present disclosure includes donor minibanks (and donor banks comprising a plurality of such donor minibanks), which donor minibanks include such cell therapy products derived from the blood samples collected from such suitable third party blood donors, as well as methods of making, administering, and using such cell therapy products (including, for example antigen-specific T cell line products, e.g., VSTs products), for preventing diseases or disorders. Thus, in various embodiments, such donor minibanks include a plurality of cell therapy products (e.g., antigen-specific T cell lines) derived from samples (e.g., mononuclear cells such as PBMCs) obtained from the donors via the methods disclosed herein, for use as prophylactic adoptive immunotherapy to prevent and/or control viral infections, diseases, and/or disorders.

In various embodiments, one or more of the cell therapy products included in the donor minibanks disclosed herein are administered to a well-matched subject in need of such a therapy based on a patient matching method. In embodiments, a plurality of such cell therapy products included in the donor minibank are administered to a well-matched subject based on a patient matching method. In embodiments, the donors utilized in constructing the donor minibanks disclosed herein are pre-screened for seropositivity and/or the donors are healthy. The present disclosure provides that these antigen-specific T cell lines are prospectively generated and then cryopreserved so that they are immediately available as an “off the shelf” product with demonstrable prophylactic utility against a virus or multiple viruses.

The present disclosure provides, in embodiments, that polyclonal VSTs may be made without requiring the presence of live viruses or recombinant DNA technologies in the manufacturing process. In embodiments, T cell populations are expanded and enriched for virus specificity with a consequent loss in alloreactive T cells. In embodiments, the cell therapy (e.g., VST) donor banks and donor minibanks are sufficiently HLA-matched to mediate antiviral effects against virally infected cells. For example, sufficiently HLA-matched indicates that at least 2 alleles are matched. In embodiments, the 2 or more alleles comprise at least 2 HLA Class I alleles. In embodiments, the 2 or more alleles comprise at least 2 HLA Class II alleles. In embodiments, the 2 or more alleles comprise at least 1 HLA Class I allele and at least 1 HLA Class II allele.

In embodiments, methods of constructing a first donor minibank of antigen-specific T cell lines comprise isolating MNCs, or having MNCs, isolated, from blood obtained from each respective donor included in the donor minibank. The blood from each donor included in the donor bank can be harvested. In embodiments, mononuclear cells (MNCs) in the harvested blood from each donor included in the donor bank are collected. MNCs and PBMCs are isolated by using the methods known by a skilled person in the art. By way of examples, density centrifugation (gradient) (Ficoll-Paque) can be used for isolating PBMCs. In other example, cell preparation tubes (CPTs) and SepMate tubes with freshly collected blood can be used for isolating PBMCs. By way of example, PBMC can comprise lymphocytes, monocytes, and dendritic cells. By way of example, lymphocytes can include T cells, B cells, and NK cells. In embodiments, the MNCs as used herein are cultured or cryopreserved. In embodiments, the process of culturing or cryopreserving the cells can include contacting the cells in culture with one or more antigens under suitable culture conditions to stimulate and expand antigen-specific T cells. In embodiments, the one or more antigen can comprise one or more viral antigen.

In embodiments, the process of culturing or cryopreserving the cells can include contacting the cells in culture with one or more epitope from one or more antigen under suitable culture conditions. In embodiments, contacting the MNCs or PBMCs with one or more antigen, or one or more epitope from one or more antigen, stimulate and expand a polyclonal population of antigen-specific T cells from each of the respective donor's MNCs or PMBCs. In embodiments, the antigen-specific T cell lines can be cryopreserved.

In embodiments, the one or more antigen can be in the form of a whole protein. In embodiments, the one or more antigen can be a pepmix comprising a series of overlapping peptides spanning part of or the entire sequence of each antigen. In embodiments, the one or more antigen can be a combination of a whole protein and a pepmix comprising a series of overlapping peptides spanning part of or the entire sequence of each antigen.

In embodiments, the culturing of the PBMCs or MNCs is in a vessel comprising a gas permeable culture surface. In one embodiment, the vessel is an infusion bag with a gas permeable portion or a rigid vessel. In one embodiment, the vessel is a GRex bioreactor. In one embodiment, the vessel can be any container, bioreactor, or the like, that are suitable for culturing the PBMCs or MNCs as described herein.

In embodiments, the PBMCs or MNCs are cultured in the presence of one or more cytokine. In embodiments, the cytokine is IL4. In embodiments, the cytokine is IL7. In embodiments, the cytokine is IL4 and IL7. In embodiments, the cytokine includes IL4 and IL7, but not IL2. In embodiments, the cytokine can be any combinations of cytokines that are suitable for culturing the PBMCs or MNCs as described herein.

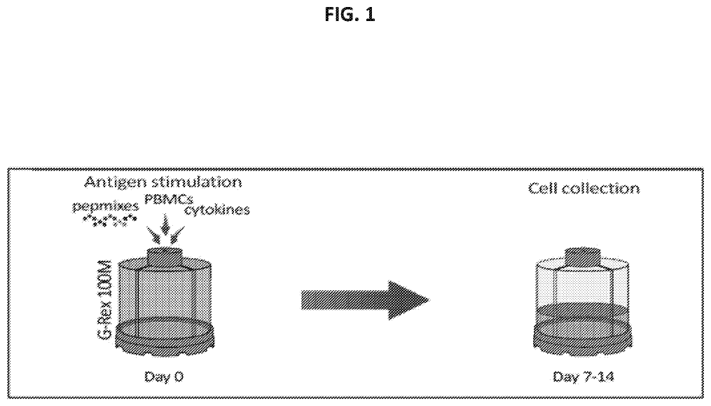

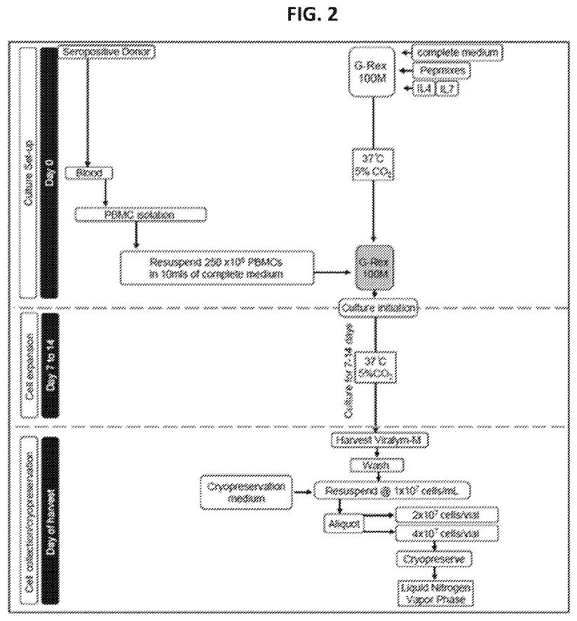

In embodiments, culturing the MNCs or PBMCs can be in the presence of at least 2, 3, 4, 5, 6, 7, 8, 9, 10, 11, 12, 13, 14, 15, 16, 17, 18, 19, 20, or more different pepmixes. Pepmixes, a plurality of peptides, comprise a series of overlapping peptides spanning part of or the entire sequence of an antigen. In embodiments, the MNCs or PBMCs can be cultured in the presence of a plurality of pepmixes. In this instance, each pepmix covers at least one antigen that is different than the antigen covered by each of the other pepmixes in the plurality of pepmixes. In embodiments, at least 2, 3, 4, 5, 6, 7, 8, 9, 10, 11, 12, 13, 14, 15, 16, 17, 18, 19, 20 or more different antigens are covered by the plurality of pepmixes. In embodiments, at least one antigen from at least 2 different viruses are covered by the plurality of pepmixes. FIG. 1 and FIG. 2 show an example of a general GMP manufacturing protocol of constructing the antigen-specific T cell lines.

In embodiments, the pepmix comprises 15 mer peptides. In embodiments, the pepmix comprises peptides that are suitable for the methods as described herein. In embodiments, the peptides in the pepmix that span the antigen overlap in sequence by 8 amino acids, 9 amino acids, 10 amino acids, 11 amino acids, 12 amino acids, 13 amino acids, 14 amino acids, 15 amino acids. In embodiments, the peptides in the pepmix that span the antigen overlap in sequence by 11 amino acids.

In embodiments, the viral antigen in the one or more pepmixes is from a virus selected from EBV, CMV, Adenovirus, BK, JC virus, HHV6, RSV, Influenza, Parainfluenza, Bocavirus, Coronavirus (e.g., SARS-CoV-2), LCMV, Mumps, Measles, human Metapneumovirus, Parvovirus B, Rotavirus, merkel cell virus, herpes simplex virus, HPV, HBV, HIV, HTLV1, HHV8, West Nile Virus, zika virus, and ebola virus. In embodiments, at least one pepmix covers an antigen from RSV, Influenza, Parainfluenza, and Human meta-pneumovirus (HMPV). In embodiments, at least one pepmix covers an antigen from EBV, CMV, BKV, and HHV6. In embodiments, at least one pepmix covers an antigen from HHV8 or HBV. In embodiments, the virus can be any suitable virus.

In embodiments, the influenza antigens can be influenza A antigen NP1. In embodiments, the influenza antigens can be influenza A antigen MP1. In embodiments, the influenza antigens can be a combination of NP1 and MP1. In embodiments, the RSV antigens can be RSV N. In embodiments, the RSV antigens can be RSV F. In embodiments, the RSV antigens can be a combination of RSV N and F. In embodiments, the hMPV antigens can be F. In embodiments, the hMPV antigens can be N. In embodiments, the hMPV antigens can be M2-1. In embodiments, the hMPV antigens can be M. In embodiments, the hMPV antigens can be a combination of F, N, M2-1, and M. In embodiments, the PIV antigens can be M. In embodiments, the PIV antigens can be HN. In embodiments, the PIV antigens can be N. In embodiments, the PIV antigens can be F. In embodiments, the PIV antigens can be a combination of M, HN, N, and F.

In embodiments, the PBMCs or MNCs are cultured in the presence of pepmixes spanning influenza A antigen NP1 and Influenza A antigen MP1, RSV antigens N and F, hMPV antigens F, N, M2-1, and M, and PIV antigens M, HN, N, and F. In embodiments, the PBMCs or MNCs are cultured in the presence of pepmixes spanning EBV antigens LMP2, EBNA1, and BZLF1, CMV antigens IE1 and pp65, adenovirus antigens Hexon and Penton, BK virus antigens VP1 and large T, and HHV6 antigens U90, Ulf, and U14. In embodiments, the antigen specific T cells are tested for antigen-specific cytotoxicity.

In other embodiments, at least one pepmix covers an antigen from EBV, CMV, adenovirus, BK, and HHV6. In embodiments, the EBV antigens are from LMP2, EBNA1, BZLF1, and a combination thereof. In embodiments, the CMV antigens are from IE1, pp65, and a combination thereof. In embodiments, the adenovirus antigens are from Hexon, Penton, and a combination thereof. In embodiments, the BK virus antigens are from VP1, large T, and a combination thereof. In embodiments, the HHV6 antigens are from U90, U11, U14, and a combination thereof.

In embodiments, at least one pepmix covers an antigen from HHV8. In embodiments, the antigens from HHV8 are selected from LANA-1 (ORF3); LANA-2 (vIRF3, K10.5); vCYC (ORF72); RTA (ORF50); vFLIP (ORF71); Kaposin (ORF12, K12); gB (ORF8); MIR1 (K3); SSB (ORF6); TS(ORF70), and a combination thereof.

In embodiments, at least one pepmix covers an antigen from HBV. In embodiments, the antigens from HBV are selected from HBV core antigen, HBV Surface Antigen, and a combination of HBV core antigen and HBV Surface Antigen.

In embodiments, the pepmix covers an antigen from SARS-CoV-2. In embodiments, the SARS-CoV-2 antigen comprises one or more antigen selected from the group consisting of (i) nsp1; nsp3; nsp4; nsp5; nsp6; nsp10; nsp12; nsp13; nsp14; nsp15; and nsp16; (ii) Spike (S); Envelope protein (E); Matrix protein (M); and Nucleocapsid protein (N); and (iii) SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14).

The present disclosure provides methods of preventing or controlling a disease or condition comprising administering to a patient one or more suitable antigen-specific T cell lines from the minibank as described herein. In embodiments, the sole criteria for qualifying the antigen-specific T cell line for administration to the patient is that the patient shares at least two HLA alleles with the donor from whom the MNCs or PBMCs used in the manufacture of the antigen-specific T cell line were isolated. In embodiments, the present disclosure includes methods for identifying the most suitable cell therapy product (e.g., antigen-specific T cell line) from a donor minibank for administration to a given patient. In embodiments, the patient has received a haematopoietic stem cell transplant. In some such embodiments, the sole criteria for qualifying the antigen-specific T cell line for administration to the patient is that the patient and the patient's haematopoietic stem cell donor share at least two matched HLA alleles with the donor from whom the MNCs or PBMCs used in the manufacture of the antigen-specific T cell line were isolated.

In embodiments, the disease prevented via the methods provided herein is a viral infection. In embodiments, the diseases prevented is associated with or caused by an immune deficiency in the subject. In embodiments, the immune deficiency is primary immune deficiency.

In embodiments, the patient is at a higher risk than an average person in the general population of contracting a viral infection or of having a latent virus reactivate. In embodiments, the viral infection or reactivation of a latent virus poses a greater risk to the patient's health compared to the risk that such an infection or reactivation would pose to an average person in the general population. In embodiments, the patient is immunocompromised. As used herein, immunocompromised means having a weakened immune system. For example, patients who are immunocompromised have a reduced ability to fight infections and other diseases. In embodiments, the patient is immunocompromised due to a treatment the patient received to treat the disease or condition or another disease or condition. In embodiments, the patient is immunocompromised due to age. In one embodiment, the patient is immunocompromised due to young age. For example, in embodiments, the patient is less than 1 year of age. In one embodiment, the patient is immunocompromised due to old age. For example, in embodiments, the patent is over 60 years of age, over 65 years of age, over 70 years of age, over 75 years of age, over 80 years of age, or over 85 years of age. In embodiments, the patient is immunocompromised due to young or old age coupled with an immune deficiency. In embodiments, the patient is in need of a transplant therapy.

The present disclosure provides methods of selecting and using a first antigen-specific T cell line from the minibank or from a minibank comprised in the donor bank, for administration in an allogeneic T cell therapy to a patient who has received or is in need of receiving transplanted material from a transplant donor in a transplant procedure. In one embodiment, the administration is for prevention of a viral infection or prevention of a disease or disorder caused by a viral infection or by reactivation of a latent virus. In one embodiment, the administration is for primary immune deficiency prior to transplant. In embodiments, the transplanted material comprises stem cells. In embodiments, the transplanted material comprises a solid organ or tissue. In embodiments, the transplanted material comprises bone marrow. In embodiments, the transplanted material comprises stem cells, a solid organ, and bone marrow.

In embodiments, the primary immune deficiency disease (PIDD) may be a genetic disorder. Exemplary PIDDs include autoimmune lymphoproliferative syndrome (ALPS), autoimmune polyglandular syndrome type 1 APS-1), BENTA disease, caspase 8 deficiency state, CARDS deficiency, chronic granulomatous disease (CGD), common variable immuonodeficiency, congenital neutropenia syndromes, CTLA4 deficiency, DOCK8 deficiency, GATA2 deficiency, glycosylation disorders, hyper-immunoglobulin E syndromes, hyper-immunoglobulin M syndromes, cytokine deficiencies, leukocyte adhesion deficiency, LRBA deficiency, PI3 kinase disease, PCLG2-associated antibody deficiency and immune dysregulation (PLAID), severe combined immunodeficiency (SCID), STAT3 dominant negative disease, STAT3 gain of function disease, WHIM syndrome, Wiskott-Aldrich syndrome, X-linked agammaglobulinemia, X-linked lymphoproliferative disease, XMEN disease, complement deficiency, selective IgA deficiency, DiGeorge syndrome, and ataxia-telangectasia. In embodiments, the patient has an immune deficiency disease that is not a PIDD, for example, an HIV infection and/or acquired immunodeficiency syndrome (AIDS).

In embodiments, the patient is administered a first antigen-specific T cell line a plurality of times. For example, in embodiments the first antigen-specific T cell line may be administered to the patient 2, 3, 4, 5, or more times. In embodiments, a second antigen-specific T cell line is administered to the patient. In embodiments, the second antigen-specific T cell line is selected from the same minibank as the first antigen specific T cell line. In embodiments, the second antigen-specific T cell line is selected from a different minibank than the minibank from which the first antigen specific T cell line was obtained. In embodiments, the second antigen specific T cell line is administered to the patient a plurality of times, e.g., 2, 3, 4, 5, or more times. In embodiments, the patient is administered a plurality of additional antigen specific T cell lines. For example, in embodiments, the methods provided herein comprise administering 2, 3, 4, 5, 6, 7, 8, 9, 10, or more different antigen-specific T cell lines. In embodiments, the antigen-specific T cell lines comprise the same antigen specificity as one another, but are generated from different donors. In embodiments, the antigen-specific T cell lines comprise different specificities and are generated from the same donor. In embodiments, the antigen-specific T cell lines comprise different specificities and are generated from different donors.

In embodiments, the methods comprise administering the polyclonal antigen specific T cell line to the subject, and then administering an antigen composition to boost the response to one or more of the viruses or antigens for which the polyclonal antigen specific T cells are specific. For example, in embodiments, the methods comprise administering an antigen composition to boost the response about 2, 3, 4, 5, 6, 7, 8, 9, 10, 11, 12, or more weeks after administration of the polyclonal antigen specific T cell line. In embodiments, the antigen composition comprises one or more peptides, or one or more whole antigens (e.g., any of the virus antigens provided herein). In some embodiments, the antigen composition comprises the pepmix or pepmixes used to produce the polyclonal antigen specific T cell line, or one or more of the antigenic peptides contained in the pepmix or pepmixes used to produce the polyclonal antigen specific T cell line. In embodiments, the antigen composition further comprises an adjuvant. Exemplary adjuvants include, but are not limited to, aluminum salts such as aluminum hydroxide ((Al(OH)₃), aluminum hydroxide gel (alum) or aluminum phosphate; salts of calcium, iron or zinc; Freund's Incomplete Adjuvant, Freund's Complete Adjuvant, Merck Adjuvant 65, toll-like receptor type 4 (TLR-4) agonists (e.g., monophosphoryl lipid A (MPL), synthetic lipid A, lipid A mimetics or analogs), aluminum salts, cytokines, saponins, muramyl dipeptide (MDP) derivatives, CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, liposomes, oil-in-water emulsions, MF59, 3DMPL, QS21, and squalene.

The present disclosure provides methods of preventing a disease or condition or a viral infection or the reactivation of a latent virus, comprising administering to a patient one or more third-party allogeneic T cell therapy, comprising administering to the patient one or more polyclonal antigen-specific T cell line. In embodiments, the T cell line comprises antigen specificity for one or more viral antigen. In embodiments, the T cell line comprises an HLA type that matches the patient's HLA type on 2 or more HLA alleles. For example, the T cell line comprises an HLA type that matches the patient's HLA type on 2, 3, 4, 5, or 6 alleles. In some embodiments, the patient has received a HSCT and the T cell line is matched on 2 or more HLA alleles with both the patient and the HSCT donor.

Inflammatory response can be detected by observing one or more symptom or sign of (i) constitutional symptoms selected from fever, rigors, headache, malaise, fatigue, nausea, vomiting, arthralgia; (ii) vascular symptoms including hypotension; (iii) cardiac symptoms including arrhythmia; (iv) respiratory compromise; (v) renal symptoms including kidney failure and uremia; and (vi) laboratory symptoms including coagulopathy and a hemophagocytic lymphohistiocytosis-like syndrome. In embodiments, inflammatory response can be detected by observing any signs that are known or common.

In embodiments, the efficacy of the prophylactic method is measured post-administration of the antigen specific T cell line. In embodiments, the efficacy of the prophylactic method is measured based on viral load in a sample from the patient. In embodiments, the efficacy of the prophylactic method is measured by monitoring viral load detectable in the peripheral blood of the patient. In embodiments, the efficacy of the prophylactic method comprises reduction or maintenance of macroscopic hematuria. In embodiments, the efficacy of the prophylactic method comprises reduction or maintenance of hemorrhagic cystitis symptoms as measured by the CTCAE-PRO or similar assessment tool that examines patient and/or clinician-reported outcomes. In embodiments, the efficacy of the prophylactic method is measured by monitoring markers of disease burden detectable in the peripheral blood/serum of the patient.

The sample is selected from a tissue sample from the patient. The sample is selected from a fluid sample from the patient. The sample is selected from cerebral spinal fluid (CSF) from the patient. The sample is selected from BAL from the patient. The sample is selected from stool from the patient.

### Exemplary Clinically Significant Viruses

Viral infections are a serious cause of morbidity and mortality after allogenic hematopoietic stem cell transplantation (allo-HSCT) or solid organ transplantation. Viral reactivation is likely to occur during the relative or absolute immunodeficiency of aplasia and during immunosuppressive therapy after allo-HSCT. Infections associated with viral pathogens including cytomegalovirus (CMV), BK virus (BKV), and adenovirus (AdV), have become increasingly problematic following allo-HSCT and are associated with significant morbidity and mortality.

Among the common infections, CMV remains the most clinically significant infection after allogeneic hematopoietic stem cell transplant (HSCT) and is also a significant infection after solid organ transplantation. Center for International Blood and Marrow Transplant Research (CIBMTR) data show that early post-transplant CMV reactivation occurs in over 30% of CMV seropositive HSCT recipients and can result in colitis, retinitis, pneumonitis, and death. Although antiviral agents including ganciclovir, valganciclovir, letermovir, foscarnet and cidofovir have been used both prophylactically and therapeutically, they are not always effective and are associated with significant toxicities including bone marrow suppression, renal toxicity, and ultimately, non-relapse mortality. Since immune reconstitution remains paramount to infection control, the adoptive transfer of ex vivo expanded/isolated CMV-specific T cells (CMVSTs) has emerged as an effective means of providing antiviral benefit.

Early immunotherapies targeting CMV focused on stem cell donor-derived T cell products, which proved both safe and effective in a series of academic Phase I/II studies spanning more than 20 years. However, the personalized nature of the therapy as well the requirement for virus-immune donors (an important issue given the benefits of using younger donors that are more likely virus-naive) have emerged as barriers that preclude broad implementation. Thus, more recently, partially HLA-matched third party-derived virus-specific T cells (VSTs), which can be prepared prospectively and banked in advance of clinical need, have been investigated as a therapeutic modality. These VSTs have proved safe and effective against a spectrum of viruses including Epstein-Barr virus, CMV, adenovirus, HHV6 and BK virus in >150 HSCT or solid organ transplant (SOT) recipients with drug-refractory infections/disease. These studies prompted interest in advancing “off the shelf” virus-specific T cells towards pivotal studies and subsequent commercialization, with the remaining questions relating to (i) the number of cell lines required to accommodate the diverse transplant population, and (ii) establishing criteria for line selection to assure clinical benefit.

In addition, the emergence of infections caused by reactivation of latent BKV, a member of the Polyomavirus family, causes severe clinical disease in HSCT patients as well as kidney transplant recipients. The primary clinical manifestation of BKV infection is hemorrhagic cystitis (BK-HC). This occurs in up to 25% of allogeneic HSCT recipients and manifests as gross hematuria with severe, often debilitating, abdominal pain requiring continuous narcotic infusions. In healthy individuals, T cell immunity defends against viruses. In allo-HSCT recipients the use of potent immunosuppressive regimens (and subsequent associated immune compromise) leaves patients susceptible to severe viral infections.

AdV can cause significant morbidity and mortality after allogeneic HSCT with known risk factors including pediatric HSCT, mismatched donors, T cell depletion, cord blood transplantation, GVHD grades III-IV and lymphopenia. Overall, younger age is associated with an increased incidence for AdV infection. Following a review of 1,738 patients transplanted at 50 centers in Europe, Voigt and colleagues reported that 1 in 3 (33%) pediatric allogeneic-HSCT recipients developed an AdV infection (defined as AdV DNA in plasma) within the first 6 months post-transplant. AdV infection can progress to severe and protracted systemic illnesses such as pneumonitis, colitis, hemorrhagic cystitis, hepatitis and encephalitis in up to 40% of the infected patients, resulting in an overall mortality from AdV infection after HSCT of between 19-83% amongst pediatric allogeneic HSCT recipients. In addition, AdV infection in pediatric allogeneic-HSCT is associated with significant medical resource utilization, as measured by duration of hospital stay. In a multicenter, multinational study of 520 pediatric allogeneic-HSCT recipients, those with AdV viremia (defined as AdV DNA in blood >1000 copies/mL) were hospitalized 22 days longer than those without AdV infection. In a separate study, the economic burden (in antiviral costs and inpatient hospital stay) of AdV infection in pediatric allogeneic HSCT recipients was estimated at $31,500 per patient compared to $1,120 in patients without AdV infection. Off-label antiviral therapies with cidofovir are ineffective and nephrotoxic. Importantly, these antivirals are virostatic and have no impact on promoting T cell immune reconstitution, which is crucial for recovery from AdV infection. Reconstitution of AdV-specific immunity remains paramount for infection clearance, and 3rd party AdV-specific T cells including Viralym-M have been successfully used to treat active AdV infection and disease.

Respiratory viral infections due to community-acquired respiratory viruses (CARVs) including respiratory syncytial virus (RSV), influenza, parainfluenza virus (PIV) and human metapneumovirus (hMPV) are detected in up to 40% of allogeneic hematopoietic stem cell transplant (allo-HSCT) recipients, in whom they may cause severe disease such as bronchiolitis and pneumonia that can be fatal. RSV induced bronchiolitis is the most common reason for hospital admission in children less than 1 year, while the Center for Disease Control (CDC) estimates that, annually, Influenza accounts for up to 35.6 million illnesses worldwide, between 140,000 and 710,000 hospitalizations, annual costs of approximately $87.1 billion in disease management in the US alone and between 12,000 and 56,000 deaths.

The present disclosure provides restoration of T cell immunity by the administration of ex vivo expanded, non-genetically modified, virus-specific T cells (VSTs) to control viral infections and eliminate symptoms for the period until the transplant patient's own immune system is restored. Without wishing to be bound by any theories, VSTs are capable of circulating for at least 6 weeks or at least 12 weeks and prophylactically prevent viral infection or prophylactically prevent reactivation of a latent virus. In embodiments, VSTs recognize and kill virus-infected cells via their native T cell receptor (TCR), which binds to major histocompatibility complex (MHC) molecules expressed on target cells that present virus-derived peptides.

In embodiments, VSTs from peripheral blood mononuclear cells (PBMCs) procured from healthy, pre-screened, seropositive donors, which are available as a partially HLA-matched “off-the-shelf” product. In embodiments, the VSTs as described herein respond to any one or more of EBV, CMV, AdV, BKV, HHV6, HHV8, hepatitis B virus (HBV), RSV, influenza, PIV, hMPV, and SARS-COV-2. In embodiments, the VSTs as described herein respond to at least EBV, CMV, AdV, BKV, and HHV6. In embodiments, the VSTs as described herein respond to HBV or HHV8. In embodiments, the VSTs as described herein respond to SARS-CoV-2. In embodiments, the VSTs as described herein respond to RSV, influenza, PIV and hMPV. In embodiments, the VSTs are designed to circulate in the recipient patient until the patient regains immunocompetence, e.g., following HSCT engraftment and immune system repopulation. Without wishing to be bound by theories, in embodiments, the VSTs and methods as described herein are “immunologic bridge therapy” that provides an immunocompromised patient with T cell immunity until the patient engrafts and can mount an endogenous immune response. In embodiments, the VSTs are designed to circulate in the recipient at least until a further administration of the VSTs, e.g., a subsequent dose of VSTs about 4, 5, 6, 7, 8, 9, 10, 11, 12, or more weeks following the previous dose. In embodiments, a peptide or whole antigen boost is administered to the patient about 2, 3, 4, 5, 6, 7, 8, 9, 10, 11, 12, or more weeks following the administration of the VSTs.

In embodiments of the disclosure, the generated antigen specific T cells are provided to an individual that has or is at risk of having a pathogenic infection, including a viral, bacterial, or fungal infection. The individual may or may not have a deficient immune system. In some cases, the individual is at risk of a viral, bacterial, or fungal infection following organ or stem cell transplant (including hematopoietic stem cell transplantation), or has cancer or has been or will be subjected to cancer treatment, for example. In some cases the individual has an acquired immune system deficiency.

The infection in the individual may be of any kind, but in specific embodiments the infection is the result of one or more viruses. The pathogenic virus may be of any kind, but in specific embodiments it is from one of the following families: Adenoviridae, Picornaviridae, Coronavirus, Herpesviridae, Hepadnaviridae, Flaviviridae, Retroviridae, Orthomyxoviridae, Paramyxoviridae, Papovaviridae, Polyomavirus, Rhabdoviridae, or Togaviridae. In embodiments, the virus produces antigens that are immunodominant or subdominant or produces both kinds. In specific cases, the virus is selected from the group consisting of EBV, CMV, Adenovirus, BK virus, HHV6, RSV, Influenza, Parainfluenza, HHV8, HBV, Bocavirus, Coronavirus (e.g., SARS-CoV-2), LCMV, Mumps, Measles, Metapneumovirus, Parvovirus B, Rotavirus, West Nile Virus, Spanish influenza, and a combination thereof.

In some aspects the infection is the result of a pathogenic bacteria, and the present invention is applicable to any type of pathogenic bacteria. Exemplary pathogenic bacteria include at least Mycobacterium tuberculosis, Mycobacterium leprae, Clostridium botulinum, Bacillus anthracis, Yersinia pestis, Rickettsia prowazekii, Streptococcus, Pseudomonas, Shigella, Campylobacter, and Salmonella.

In some aspects the infection is the result of a pathogenic fungus, and the present invention is applicable to any type of pathogenic fungus. Exemplary pathogenic fungi include at least Candida, Aspergillus, Cryptococcus, Histoplasma, Pneumocystis, or Stachybotrys. In embodiments, viral antigens can be any antigens that are suitable for the use as described in the present disclosure.

### Generation of Pepmix Libraries

In embodiments of the invention, a library of peptides is provided to PBMCs ultimately to generate antigen specific T cells. The library in particular cases comprises a mixture of peptides (“pepmixes”) that span part or all of the same antigen. Pepmixes utilized in the invention may be from commercially available peptide libraries made up of peptides that are 15 amino acids long and overlapping one another by 11 amino acids, in certain aspects. In some cases, they may be generated synthetically. Examples include those from JPT Technologies (Springfield, Va.) or Miltenyi Biotec (Auburn, Calif.). In particular embodiments, the peptides are at least 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and in specific embodiments there is overlap of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length, for example.

In embodiments, the amino acids as used in the pepmixes have at least 85%, at least 86%, at least 87%, at least 88%, at least 89%, at least 90%, at least 91%, at least 92%, at least 93%, at least 94%, at least 95%, at least 96%, at least 97%, at least 98%, at least 99, at least 99.9% purity, inclusive of all ranges and subranges therebetween. In embodiments, the amino acids as used here in the pepmixes have at least 90% purity.

The mixture of different peptides may include any ratio of the different peptides, although in embodiments each particular peptide is present at substantially the same numbers in the mixture as another particular peptide. The methods of preparing and producing pepmixes for multiviral antigen-specific T cells with broad specificity is described in US2018/0187152, which is incorporated by reference in its entirety.

### Polyclonal Virus-Specific T Cell Compositions

The present disclosure includes polyclonal virus-specific T cell compositions, generated from seropositive donors (e.g., selected via the donor selection methods disclosed herein), with specificity against clinically significant viruses. In embodiments, the clinically significant viruses can include but are not limited to EBV, CMV, AdV, BKV and HHV6. In embodiments, the clinically significant viruses include but are not limited to RSV, influenza, parainfluenza virus, and HMPV. In embodiments, the clinically significant virus is HBV. In embodiments, the clinically significant virus is HHV8. In embodiments, the clinically significant virus is SARS-CoV-2.

The present disclosure provides a composition comprising a polyclonal population of antigen specific T cells. In embodiments, the polyclonal population of antigen specific T cells can recognize a plurality of viral antigens. In embodiments, the polyclonal population of antigen specific T cells can recognize two or more, or a plurality, of viral antigens from a single virus. For example, in embodiments, the polyclonal population of antigen specific T cells can recognize two or more, or a plurality, of viral antigens from HHV8, HBV, AdV, CMV, BKV, EBV, HHV6, JCV, RSV, Influenza, PIV, HPMV, or SARS-CoV-2. In embodiments, the polyclonal population of antigen specific T cells can recognize two or more, or a plurality, of viral antigens from more than one virus, e.g., from 2, 3, 4, 5, 6, or more different viruses.

In embodiments, the plurality of viral antigens can comprise at least one first antigen from parainfluenza virus type 3 (PIV-3). In embodiments, the plurality of viral antigens can comprise at least one second antigen from one or more second virus. In embodiments, polyclonal virus-specific T cell compositions have specificity against any clinically significant or relevant viruses. For example, polyclonal virus-specific T cell compositions can comprise viral antigens selected from CMV, BKV, EBV, AdV, HHV6, HHV8, HBV, JCV, PIV3, RSV, HMPV, Influenza, and SARS-CoV-2, or any combination thereof.

In embodiments, the present disclosure provides a polyclonal population of antigen specific T cells that recognize a plurality of viral antigens comprising at least one antigen from each of parainfluenza virus type 3 (PIV-3) respiratory syncytial virus, Influenza, and human metapneumovirus, as well as donor minibanks as described herein containing a plurality of cell lines containing such antigen specific T cells. In embodiments, the present disclosure provides a polyclonal population of antigen specific T cells that recognize a plurality of viral antigens comprising the plurality of viral antigens comprise at least two antigens from each of parainfluenza virus type 3 (PIV-3) respiratory syncytial virus, Influenza, and human metapneumovirus, as well as donor minibanks as described herein containing a plurality of cell lines containing such antigen specific T cells.

In embodiments, the plurality of antigens comprise PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N. In embodiments, the plurality of antigens can be selected from any of PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N.

In embodiments, the first antigen can be PIV-3 antigen M. In embodiments, the first antigen can be PIV-3 antigen HN. In embodiments, the first antigen can be PIV-3 antigen N. In embodiments, the first antigen can be PIV-3 antigen F. In embodiments, the first antigen can be any combinations of PIV-3 antigen M, PIV-3 antigen HN, PIV-3 antigen N, and PIV-3 antigen F. In embodiments, the composition can comprise 1 first antigen. In embodiments, the composition can comprise 2 first antigens. In embodiments, the composition can comprise 3 first antigens. In embodiments, the composition can comprise 4 first antigens. In embodiments, the 4 first antigens can comprise PIV-3 antigen M, PIV-3 antigen HN, PIV-3 antigen N, and PIV-3 antigen F.

In embodiments, the one or more second virus can be respiratory syncytial virus (RSV). In embodiments, the one or more second virus can be Influenza. In embodiments, the one or more second virus can be human metapneumovirus (hMPV). In embodiments, the one or more second virus can comprises respiratory syncytial virus (RSV), Influenza, and human metapneumovirus. In embodiments, the one or more second virus can consist of respiratory syncytial virus (RSV), Influenza, and human metapneumovirus. In embodiments, the one or more second virus can be selected from any suitable viruses as described herein.

In embodiments, the composition can comprise two or three second viruses. In embodiments, the composition can comprise three second viruses. In embodiments, the three second viruses can comprise influenza, RSV, and hMPV. In embodiments, the composition comprise at least two second antigens per each second virus. In embodiments, the composition comprises 1 second antigen. In embodiments, the composition comprises 2 second antigens. In embodiments, the composition comprises 3 second antigens. In embodiments, the composition comprises 4 second antigens. In embodiments, the composition comprises 5 second antigens. In embodiments, the composition comprises 6 second antigens. In embodiments, the composition comprises 7 second antigens. In embodiments, the composition comprises 8 second antigens. In embodiments, the composition comprises 9 second antigens. In embodiments, the composition comprises 10 second antigens. In embodiments, the composition comprises 11 second antigens. In embodiments, the composition comprises 12 second antigens. In embodiments, the composition comprises any numbers of second antigens that would be suitable for the compositions as described herein.

In embodiments, the second antigen can be influenza antigen NP1. In embodiments, the second antigen can be influenza antigen MP1. In embodiments, the second antigen can be RSV antigen N. In embodiments, the second antigen can be RSV antigen F. In embodiments, the second antigen can be hMPV antigen M. In embodiments, the second antigen can be hMPV antigen M2-1. In embodiments, the second antigen can be hMPV antigen F. In embodiments, the second antigen can be hMPV antigen N. In embodiments, the second antigen can be any combinations of influenza antigen NP1, influenza antigen MP1, RSV antigen N, RSV antigen F, hMPV antigen M, hMPV antigen M2-1, hMPV antigen F, and hMPV antigen N.

In embodiments, the second antigen comprises influenza antigen NP1. In embodiments, the second antigen comprises influenza antigen MP1. In embodiments, In embodiments, the second antigen comprises both influenza antigen NP1 and influenza antigen MP1. In embodiments, the second antigen comprises RSV antigen N. In embodiments, the second antigen comprises RSV antigen F. In embodiments, the second antigen comprises both RSV antigen N RSV antigen F.

In embodiments, the second antigen comprises hMPV antigen M. In embodiments, the second antigen comprises hMPV antigen M2-1. In embodiments, the second antigen comprises hMPV antigen F. In embodiments, the second antigen comprises hMPV antigen N. In embodiments, the second antigen comprises combinations of hMPV antigen M, hMPV antigen M2-1, hMPV antigen F, and hMPV antigen N.

In embodiments, the second antigen comprises each of influenza antigen NP1, influenza antigen MP1, RSV antigen N, RSV antigen F, hMPV antigen M, hMPV antigen M2-1, hMPV antigen F, hMPV antigen N. In embodiments, the plurality of antigens comprise PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N. In embodiments, the plurality of antigens consist of PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N. In embodiments, the plurality of antigens consist essentially of PIV-3 antigen M, PIV-3 antigen HN, PIV-3 antigen N, PIV-3 antigen F, influenza antigen NP1, influenza antigen MP1, RSV antigen N, RSV antigen F, hMPV antigen M, hMPV antigen M2-1, hMPV antigen F, and hMPV antigen N. In embodiments, the second antigen can comprise any suitable antigens for the compositions as described herein.

In embodiments, the clinically significant viruses can include but are not limited to HHV8. In embodiments, the viral antigens span immunogenic antigens from HHV8. In embodiments, the antigens from HHV8 are selected from LANA-1 (ORF3); LANA-2 (vIRF3, K10.5); vCYC (ORF72); RTA (ORF50); vFLIP (ORF71); Kaposin (ORF12, K12); gB (ORFS); MIR1 (K3); SSB (ORF6); TS(ORF70), and a combination thereof.

In embodiments, the clinically significant viruses can include but are not limited to HBV. In embodiments, the viral antigens span immunogenic antigens from HBV. In embodiments, the antigens from HBV are selected from (i) HBV core antigen, (ii) HBV Surface Antigen, and (iii) HBV core antigen and HBV Surface Antigen.

In embodiments, the clinically significant viruses can include but are not limited to a coronavirus. In embodiments, the coronavirus is a α-coronavirus (α-CoV). In embodiments, the coronavirus is a β-coronavirus (β-CoV). In embodiments, the β-CoV is selected from SARS-CoV, SARS-CoV-2, MERS-CoV, HCoV-HKU1, and HCoV-OC43. In embodiments, the coronavirus is SARS-CoV-2. In embodiments, the SARS-CoV-2 antigen comprises one or more antigen selected from the group consisting of (i) nsp1; nsp3; nsp4; nsp5; nsp6; nsp10; nsp12; nsp13; nsp14; nsp15; and nsp16; (ii) Spike (S); Envelope protein (E); Matrix protein (M); and Nucleocapsid protein (N); and (iii) SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14).

In embodiments, the antigen specific T cells in the compositions can be generated by contacting peripheral blood mononuclear cells (PBMCs) with a plurality of pepmix libraries. In embodiments, each pepmix library contains a plurality of overlapping peptides spanning at least a portion of a viral antigen. In embodiments, at least one of the plurality of pepmix libraries spans a first antigen from PIV-3. In embodiments, at least one additional pepmix library of the plurality of pepmix libraries spans each second antigen.

In embodiments, the antigen specific T cells can be generated by contacting T cells with dendritic cells (DCs) nucleofected with at least one DNA plasmid. In embodiments, the DNA plasmid can encode the PIV-3 antigen. In embodiments, the at least one DNA plasmid encodes each second antigen. In embodiments, the plasmid encodes at least one PIV-3 antigen and at least one of the second antigens. In embodiments, the compositions as described herein comprise CD4+T-lymphocytes and CD8+T-lymphocytes. In embodiments, the compositions comprise antigen specific T cells expressing αβT cell receptors. In embodiments, the compositions comprise MHC-restricted antigen specific T cells.

In embodiments, the antigen specific T cells can be cultured ex vivo in the presence of both IL-7 and IL-4. In embodiments, the multivirus antigen specific T cells have expanded sufficiently within 9 days, 10 days, 11 days, 12 days, 13 days, 14 days, 15 days, 16 days, 17 days, 18 days, 19 days, 20 days inclusive of all ranges and subranges therebetween, of culture such that they are ready for administration to a patient. In embodiments, the multivirus antigen specific T cells have expanded sufficiently within any number of days that are suitable for the compositions ad described herein.

The present disclosure provides compositions comprising antigen specific T cells that exhibit negligible alloreactivity. In embodiments, the compositions comprising antigen specific T cells that exhibit less activation induced cell death of antigen-specific T cells harvested from a patient than corresponding antigen-specific T cells harvested from the same patient. In embodiments, the compositions are not cultured in the presence of both IL-7 and IL-4. In embodiments, the compositions comprising antigen specific T cells exhibit viability of greater than 70%.

In embodiments, the compositions are negative for bacteria and fungi for at least 1 days, at least 2 days, at least 3 days, at least 4 days, at least 5 days, at least 6 days at least 7 days, at least 8 days, at least 9 days, at least 10 days, in culture. In embodiments, the composition is negative for bacteria and fungi for at least 7 days in culture. In embodiments, the compositions exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In embodiments, the compositions exhibit less than 5 EU/ml of endotoxin. In embodiments, the compositions are negative for mycoplasma.

In embodiments, the pepmixes used for constructing the polyclonal population of antigen specific T cells are chemically synthesized. In embodiments, the pepmixes are optionally >10%, >20%, >30%, >40%, >50%, >60%, >70%, >80%, >90%, inclusive of all ranges and subranges therebetween, pure. In embodiments, the pepmixes are optionally >90% pure.

In embodiments, the antigen specific T cells are Th1 polarized. In embodiments, the antigen specific T cells are able to lyse viral antigen-expressing targets cells. In embodiments, the antigen specific T cells are able to lyse other suitable types of antigen-expressing targets cells. In embodiments, the antigen specific T cells in the compositions do not significantly lyse non-infected autologous target cells. In embodiments, the antigen specific T cells in the compositions do not significantly lyse non-infected autologous allogenic target cells.

The present disclosure provides pharmaceutical compositions comprising any compositions formulated for intravenous delivery (e.g., a pharmaceutical composition comprising an antigen-specific T cell line described herein formulated for intravenous delivery). In embodiments, the compositions are negative for bacteria for at least 1, at least 2, at least 3, at least 4, at least 5, at least 6, at least 7, at least 8 days, at least 9 days, at least 10 days, in culture. In embodiments, the compositions are negative for bacteria for at least 7 days in culture. In embodiments, the compositions are negative for fungi for at least 1, at least 2, at least 3, at least 4, at least 5, at least 6, at least 7, at least 8 days, at least 9 days, at least 10 days, in culture. In embodiments, the compositions are negative for fungi for at least 7 days in culture.

The present pharmaceutical compositions exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, or less than 10 EU/ml of endotoxin. In embodiments, the present pharmaceutical compositions are negative for mycoplasma.

The present disclosure provides methods of lysing a target cell comprising contacting the target cell with the compositions or pharmaceutical compositions as described herein (e.g., an antigen-specific T cell line or a pharmaceutical composition comprising such a T cell line formulated for intravenous delivery). In embodiments, the contacting between the target cell and the compositions or pharmaceutical compositions occurs in vivo in a subject. In embodiments, the contacting between the target cell and the compositions or pharmaceutical compositions occurs in vivo via administration of the antigen specific T cells to a subject. In embodiments, the subject is a human.

The present disclosure provides methods of controlling or preventing a viral infection comprising administering to a subject in need thereof the compositions or the pharmaceutical compositions as described herein (e.g., an antigen-specific T cell line or a pharmaceutical composition comprising such a T cell line formulated for intravenous delivery). In embodiments, the amount of antigen specific T cells that are administered range between 5×10³ and 5×10⁹ antigen specific T cells/m², 5×10⁴ and 5×10⁸ antigen specific T cells/m², 5×10⁵ and 5×10⁷ antigen specific T cells/m², 5×10⁴ and 5×10⁸ antigen specific T cells/m², 5×10⁶ and 5×10⁹ antigen specific T cells/m², inclusive of all ranges and subranges therebetween. In embodiments, the antigen specific T cells are administered to the subject. In embodiments, the subject is immunocompromised. In embodiments, the subject has acute myeloid leukemia. In embodiments, the subject has acute lymphoblastic leukemia. In embodiments, the subject has chronic granulomatous disease.

In embodiments, the subject can have one or more medical conditions. In embodiments, the subject receives a matched related donor transplant with reduced intensity conditioning prior to receiving the antigen specific T cells. In embodiments, the subject receives a matched unrelated donor transplant with myeloablative conditioning prior to receiving the antigen specific T cells. In embodiments, the subject receives a haplo-identical transplant with reduced intensity conditioning prior to receiving the antigen specific T cells. In embodiments, the subject receives a matched related donor transplant with myeloablative conditioning prior to receiving the antigen specific T cells. In embodiments, the subject has received a solid organ transplantation. In embodiments, the subject has received chemotherapy. In embodiments, the subject has an HIV infection and/or AIDS. In embodiments, the subject has a genetic immunodeficiency, e.g., a primary immune deficiency disease (PIDD). In embodiments, the subject has received an allogeneic stem cell transplant. In embodiments, the subject has more than one medical conditions as described in this paragraph. In embodiments, the subject has all medical conditions as described in this paragraph. In embodiments, the subject is immunocompromised due to age (e.g., the subject is elderly, for example, is over 60, over 65, over 70, over 75, or over 80 years of age; or is young, e.g., is under 1 year, under 6 months, under 3 months, or under 1 month of age). In embodiments, the subject is immunocompromised due to age in addition to one or more medical conditions described herein.

In embodiments, the composition as described herein is administered to the subject a plurality of times. In embodiments, the composition as described herein is administered to the subject more than one time. In embodiments, the composition as described herein is administered to the subject more than two times. In embodiments, the composition as described herein is administered to the subject more than three times. In embodiments, the composition as described herein is administered to the subject more than four times. In embodiments, the composition as described herein is administered to the subject more than five times. In embodiments, the composition as described herein is administered to the subject more than six times. In embodiments, the composition as described herein is administered to the subject more than seven times. In embodiments, the composition as described herein is administered to the subject more than eight times. In embodiments, the composition as described herein is administered to the subject more than nine times. In embodiments, the composition as described herein is administered to the subject more than ten times. In embodiments, the composition as described herein is administered to the subject a number of times that are suitable for the subjects. In embodiments, the composition is administered to the subject in periodic doses as provided herein, for the duration of the period that the subject is at high risk of a viral infection. In embodiments, the composition is administered to the subject in periodic doses as provided herein, for the duration of the period that the subject is immunocompromised.

In embodiments, the administration of the composition effectively prevents a viral infection in the subject and/or prevents a reactivation of a latent virus in a subject. In embodiments, the administration of the composition effectively controls a viral infection in a subject, or effectively controls a reactivation of a latent virus in a subject, wherein the subject did not have an active infection or a reactivation with respect to that virus at the time that the composition was administered. For example in embodiments, the subject does not have viremia or viruria or otherwise detectable virus with respect to a given virus, and is prophylactically administered a composition provided herein, wherein the subject subsequently becomes exposed to and/or infected with and/or reactivates the given virus, and wherein the prophylactic administration of the composition prevents the infection, controls the infection, resolves the infection, and/or prevents serious disease or complications that otherwise result from the infection. In embodiments, the viral infection is parainfluenza virus. In embodiments, the viral infection is parainfluenza virus type 3. In embodiments, the viral infection is RSV In embodiments, the viral infection is Influenza. In embodiments, the viral infection is HMPV. In embodiments, the viral infection is HHV8. In embodiments, the viral infection is HBV. In embodiments, the viral infection is BKV. In embodiments, the viral infection is CMV. In embodiments, the viral infection is EBV. In embodiments, the viral infection is HHV6. In embodiments, the viral infection is AdV. In embodiments, the viral infection is SARS-CoV-2.

In embodiments, the present disclosure provides pharmaceutical compositions comprising the compositions as described herein formulated for intravenous delivery. In embodiments, the composition as described herein is negative for bacteria. In embodiments, the composition as described herein is negative for fungi. In embodiments, the composition as described herein is negative for bacteria or fungi for at least 1 days, at least 2 days, at least 3 days, at least 4 days, at least 5 days, at least 6 days, at least 7 days, at least 8 days, at least 9 days, at least 10 days, in culture. In embodiments, the composition as described herein is negative for bacteria or fungi for at least 7 days in culture.

In embodiments, the pharmaceutical compositions formulated for intravenous delivery exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, or less than 10 EU/ml of endotoxin. In embodiments, the pharmaceutical compositions formulated for intravenous delivery are negative for mycoplasma.

The foregoing discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

### EXAMPLES

### Example 1. Construction of a Donor Bank of CMV-Specific VST (CMVST)

A clinical trial was conducted using third party T cells to treat CMV—a ubiquitous virus that remains a major cause of post-transplant morbidity and mortality.

Selection of donors for CMVST generation: To ensure that a clinically effective line could be provided for the majority of the allogeneic HSCT patient population, the HLA types of 666 allogeneic HSCT recipients treated in the Houston region (Houston Methodist or Texas Children's Hospital) were analyzed, which has a diverse ethnic make-up that is similar to the United States as a whole. These HSCT recipient HLAs were then compared with the HLA types of a pool of diverse, healthy, eligible CMV seropositive donors. In an initial step a healthy donor was identified whose HLA profile accommodated the greatest number of patients with a CMVST product. This donor was removed from the general donor pool; all patients accommodated by this donor were also removed from the unmatched patient population. Subsequently these steps were repeated with a second, third, etc. donor, each time identifying the donor who best covered the remaining patients and then removed both the donor and accommodated patients from further consideration, until a panel has been generated that covered at least 95% of the patients analyzed. This procedure was then repeated a second time to ensure that patients would have more than one potential donor option. Using this model, it was found that only 8 well-selected donors would provide >95% of the patient population with a T cell product that was matched on at least 2 HLA antigens; further increasing the donor pool would not significantly increase the number of matches. Eight of these donors were then selected with the goal to provide coverage suitable CMVST line (2:2 shared HLA antigens) with confirmed CMV activity to 2:95% of this diverse population of allogeneic HSCT recipients.

Third-party CMVST bank preparation: All donors gave written informed consent on an IRB approved protocol and met blood bank eligibility criteria. For manufacturing, a unit of blood was collected by peripheral blood draw and PBMCs isolated by ficoll gradient. 10×106 PBMCs were seeded in a G-Rex 5 bioreactor (Wilson Wolf, Minneapolis, Minn.), which includes a bottom comprised of gas permeable material and a body that houses media at a height of up 10 cm, and cultured in T cell media [Advanced RPMI 1640 (HyClone Laboratories Inc. Logan, Utah), 45% Click's (Irvine Scientific, Santa Ana, Calif.), 2 mM GlutaMAX™] TM-I (Life Technologies Grand Island, N.Y.), and 10% Fetal Bovine Serum (Hyclone)] containing 800 U/ml IL4 and 20 ng/ml IL7 (R&D Systems, Minneapolis, Minn.) and IE1, pp65 pepmixes (2 ng/peptide/ml) (JPT Peptide Technologies Berlin, Germany). On day 9-12 post initiation T cells were harvested, counted and restimulated with autologous pepmix-pulsed irradiated PBMCs [1:4 effector:target (E:T)—4×105 CMVSTs: 1.6×106 irradiated PBMCs/cm2] with IL4 (800 U/ml) and IL7 (20 ng/ml) in a G-Rex-100M. On day 3-4 of culture, the cells were fed with 200 ng/ml IL2 (Prometheus Laboratories, San Diego, Calif.), and 9-12 days post second stimulation, T cells were harvested for cryopreservation. At the time of cryopreservation, each line was microbiologically tested, immunophenotyped [CD3, CD4, CD8, CD14, CD16, CD19, CD25, CD27, CD28, CD45, CD45RA, CD56, CD62L CD69, CD83, HLADR and 7AAD (Becton Dickinson, Franklin Lakes, N.J.)], and evaluated for virus specificity by IFNγ enzyme-linked immunospot (ELISpot) assay. A cell line was defined as “reactive” when the frequency of reactive cells, as measured by IFNγ ELISpot assay, was >30 spot-forming cells (SFC)/2×105 input viral specific T cells.

Clinical trial design: This was a single center Phase I study (NCT02313857) conducted under an IND from the Food and Drug Administration (FDA) and approved by the Baylor College of Medicine Institutional Review Board (IRB). The study was open to allogeneic HSCT recipients with CMV infections or disease that had persisted for at least 7 days despite standard therapy defined as treatment with ganciclovir, foscamet, or cidofovir. Exclusion criteria included treatment with prednisone (or equivalent) 2:0.5 mg/kg, respiratory failure with oxygen saturation of <90% on room air, other uncontrolled infections, and active GVHD grade II. Patients who received ATG, Campath, other T cell immunosuppressive monoclonal antibodies, or a donor lymphocyte infusion (DLI) within 28 days of the proposed administration date were also excluded from participation. Patients initially gave their consent to search for a suitable VST line (with 2:2 shared HLA antigens), and if available and if patients met eligibility criteria, they could be enrolled on the treatment portion of the study. Each patient received a single intravenous infusion of 2×107 partially HLA-matched VSTs/m 2 with the option to receive a second infusion after 4 weeks and additional infusions at bi-weekly intervals thereafter. Therapy with standard antiviral medications could be administered at the discretion of the treating physician.

Safety endpoints: The primary objective of this pilot study was to determine the safety of CMVSTs in HSCT recipients with persistent CMV infections/disease. Toxicities were graded by the NCI Common Terminology Criteria for Adverse Events (CTCAE), Version 4.X. Safety endpoints included acute GvHD grades III-IV within 42 days of the last CMVST dose, infusion-related toxicities within 24 hours of infusion or grades 3-5 non-hematologic adverse events related to the T cell product within 28 days of the last CMVST dose and not attributable to a pre-existing infection, the original malignancy or pre-existing co-morbidities. Acute and chronic GVHD, if present, were graded according to standard clinical definitions.1,2 The study was monitored by the Dan L. Duncan Cancer Center Data Review Committee.

Assessment of outcomes: CMV loads in peripheral blood were monitored by quantitative PCR (qPCR) in Clinical Laboratory Improvement Amendments (CLIA)-approved laboratories. A complete response (CR) of the virus to treatment was defined as a decrease in viral load to below the threshold of detection by qPCR and resolution of clinical signs and symptoms of tissue disease (if present at baseline). A partial response (PR) was defined as a decrease in viral load of at least 50% from baseline. Clinical and virological responses were assigned at week 6 post CMVST infusion.

Immune Monitoring: ELISpot analysis was used to determine the frequency of circulating T cells that secreted IFNγ in response to CMV antigens and peptides. Clinical samples were collected prior to and at weeks 1, 2, 3, 4, 6 and 12 post-infusion. As a positive control, PBMCs were stimulated with Staphylococcal Enterotoxin B (1 μg/ml) (Sigma-Aldrich Corporation, St Louis, Mo.). IE1 and pp65 pepmixes (JPT Technologies, Berlin, Germany), diluted to 1000 ng/peptide/ml, were used to track donor-derived CMVSTs post-infusion. When available, peptides representing known epitopes (Genemed Synthesis Inc., San Antonio, Tex. diluted to 1250 ng/ml) were also used in ELISpot assays. For ELISpot analyses, PBMCs were resuspended at 5×10⁶/ml in T cell medium and plated in 96 well ELISpot plates. Each condition was run in duplicate. After 20 hours of incubation, plates were developed as previously described, dried overnight at room temperature in the dark, and then sent to Zellnet Consulting (New York, N.Y.) for quantification. Interferon-γ (spot-forming cells (SFC) and input cell numbers were plotted, and the frequency of T cells specific for each antigen was expressed as specific SFC per input cell numbers.

Statistical Analysis: Descriptive statistics were calculated to summarize data. Antiviral responses were summarized, and the response rate was estimated along with exact 95% binomial confidence intervals. Viral load and T cell frequency data were plotted to graphically illustrate the patterns of immune responses over time. Comparisons of changes in viral load and T cell frequency pre- and post-infusion were performed using Wilcoxon signed-ranks test. Data were analyzed with SAS system (Cary, N.C.) version 9.4 and R version 3.2.1. P-values <0.05 were considered statistically significant.

Results

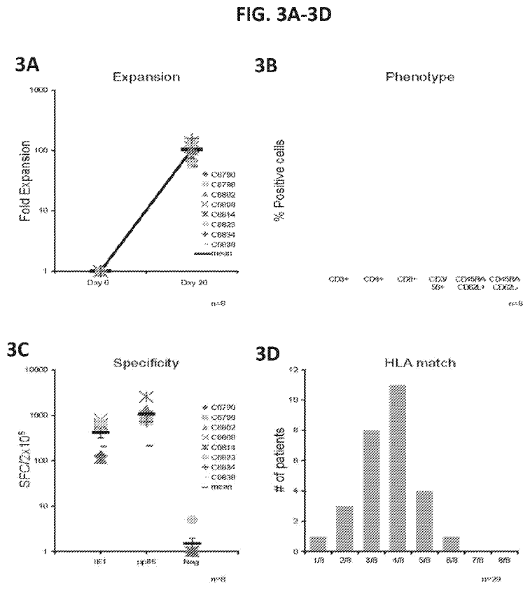

Third party CMVST bank: A bank of CMVSTs was generated from 8 CMV seropositive donors chosen to represent the diverse HLA profile of the transplant population (Table 1). A median of 7.7×10⁸ PBMCs (range 4.6-8.8×10⁸) were isolated from a single blood draw (median of 425 ml). To expand CMVSTs, PBMCs were exposed to pepmixes spanning pp65 and IE1 and over 20 days in culture a mean fold expansion of 102±12 (FIG. 3A) was achieved. The resulting cells were almost exclusively CD3+(99.3±0.4%), comprising both CD4+(21.3±7.5%) and CD8+(74.7±7.8%) subsets that expressed central CD45RA−/62L+(58.5±4.8%) and effector CD45RA−/62L− (35.3±4.6%) memory markers (FIG. 3B). All 8 lines were reactive against the stimulating CMV antigens (IE1 419±100 SFC/2×10⁵ and pp65 1069±230, FIG. 3C). Table 1 summarizes the characteristics of the cell lines. Of these 8 lines, 6 products were administered to 10 treated study patients.

Screening: 29 allogeneic HSCT recipients with CMV infections were referred by their primary BMT providers for study participation, and from a bank of 8 lines, a suitable product (minimum 2/8 HLA match threshold) was identified for infusion in 28 cases (96.6%; 95% CI: 82.2%-99.9%). A 2/8 HLA match threshold was established based on retrospective analysis performed on previous third party study which demonstrated clinical benefit associated with the administration of such HLA-matched products. HLA class I or class II matching did not appear to influence outcome. Of note, on the current study, most products were matched at >4 antigens (FIG. 1D). Of the 28 patients with available lines, 17 patients did not receive cells because they responded to standard antiviral treatment and one patient was ineligible due to a recent DLI.

Characteristics of treated patients: The characteristics of the 10 patients (pediatric n=7 and adults n=3) treated for persistent infections are summarized in Table 2 and included 2 African-American recipients, 3 patients of white Hispanic origin and 5 non-Hispanic Caucasian recipients. Three of the 10 patients had confirmed virus-associated disease [CMV retinitis (n=1), diarrhea attributed to CMV colitis (n=2)]. CMVSTs (matching at 2-6/8 HLA antigens) were administered between days 46 and 365 (median day 133) post-transplant. Seven patients had infections that were refractory to standard antiviral treatment for a median of 24 days (mean of 48 days; range 14 to 211 days), and 3 of the patients harbored viruses with mutations that conferred resistance to conventional antivirals. Prior to immunotherapeutic intervention, 6 of these patients had experienced severe adverse events (SAEs) associated with conventional antivirals that included acute kidney injury (n=4), foscarnet-induced renal tubulopathy (n=1) and severe foscarnet-associated pancreatitis (n=1), which in 3 cases precluded further treatment with any conventional drugs.

Clinical safety: All infusions were well tolerated. Except for one patient who developed a transient isolated fever 8 hours after infusion, no immediate toxicities were observed. One patient developed a mild maculopapular rash on his trunk, which appeared suggestive of a viral exanthem and spontaneously resolved within a few days without topical or systemic treatment. No cases of cytokine release syndrome (CRS) or other toxicities related to the infused CMVSTs were observed, and none of the patients developed graft failure, autoimmune hemolytic anemia or transplant associated microangiopathy. Patients were followed for 6 weeks for acute GvHD and 12 months for chronic GvHD. Despite the HLA disparity between the patients and the infused cells, none of the patients developed recurrent or de novo acute or chronic GvHD post treatment (Table 3), including 3 patients with a prior history [grade II (n=2) or III (n=1)] of acute GvHD.

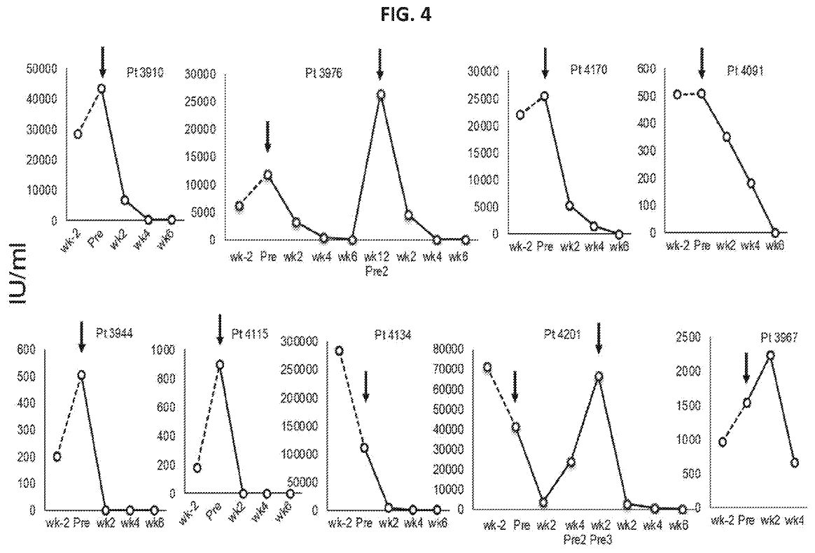

Clinical Responses: All 10 infused patients responded to CMVSTs with 7 CRs and 3 PRs, for a cumulative response rate of 100% (95% CI: 69.2-100.0%) by week 6. The average plasma viral load reduction at week 6 was 89.8% (+/−21.4%). FIG. 4 summarizes the virological outcomes of all treated patients based on sequential viral load measurements. Of note, clinical benefit was achieved not only in patients with refractory infections, but also in the 3 individuals with tissue disease [CMV retinitis (n=1), diarrhea attributed to CMV colitis (n=2)].

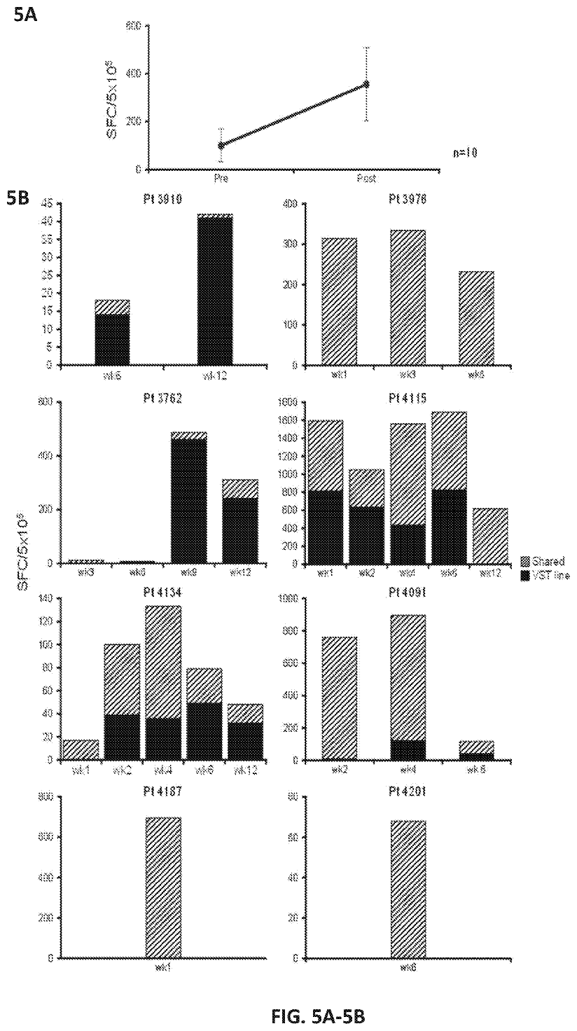

Eight patients received a single infusion of CMVSTs, 1 patient (3976) had 2 infusions and 1 (4201) had 3 infusions of CMVSTs. Patient 3976 had a CR at week 6, but relapsed with increasing virus loads at week 10. Eighty days after the first infusion, he received a second infusion with the same CMVST line that resulted in a sustained CR. Patient 4201 received a second infusion of the same CMVSTs 28 days after the initial administration but failed to respond and hence, 2 weeks later was administered a third infusion with a different CMVST line and achieved a sustained CR. The clinical and virological responses achieved in these patients were associated with an increase in virus-reactive CMVSTs in 8 of the 10 treated patients [increase from mean 126±84 SFC pre-infusion to peak of 443±178 per 5×10⁵ PBMCs (p=0.023; FIG. 5A)].

T cell persistence: To evaluate if the CMVST infusions contributed to the protective effects seen in these patients and to evaluate the in vivo longevity of these partially HLA-matched VSTs, the specificity of CMVSTs were examined in patient PBMCs before and after infusion using HLA-restricted epitope peptides restricted to the line infused. Functional T cells of confirmed third-party origin were detected in 5 patients for whom HLA-restricting peptide reagents were available, which persisted for up to 12 weeks; in all 8 patients antiviral responses restricted by the HLA alleles shared between the patient and the CMVST line (FIG. 5B) were observed. Thus, it was inferred that the infused CMVSTs induced an antiviral effect resulting in the control of CMV infections.

In the Phase I trial, third party CMVSTs were administered to treat CMV infections/disease in allogeneic HSCT recipients who had failed at least 14 day of treatment with ganciclovir and/or foscarnet or could not tolerate standard antiviral medications. Notable exclusion criteria were patients with active GvHD or receiving corticosteroids at moderate or high doses. A bank of CMVSTs was generated from just 8 healthy donors, which were carefully selected based on their HLA profile to provide broad coverage to a racially and ethnically diverse allogeneic HSCT patient population. Indeed, of the 29 patients screened for study participation, a suitable line (minimum 2 shared HLA antigen threshold) for 28 (96.6%; 95% CI: 82.2-99.9%) was identified, attesting to the feasibility of providing broad patient coverage with a small, well-chosen cell bank. Of these 28 patients, 10 from diverse backgrounds (2 African-American, 3 of white Hispanic origin and 5 non-Hispanic Caucasian) were treated and all achieved virological and clinical benefit, without experiencing acute or chronic GvHD or other toxicities. This was notable, since 6 had previously experienced serious adverse events including acute kidney injury, renal tubulopathy and pancreatitis, related to conventional antivirals. This Phase I trial showcases the safety and clinical benefit associated with the administration of 3^{rd} party virus-reactive T cells, sourced from a small and carefully designed donor bank, for the treatment of refractory CMV infections.

Despite decreasing rates of disease in recent decades, CMV remains a major cause of morbidity after allogeneic HSCT; in a recent CIBMTR report where data from 9469 patients [transplanted from 2003-2010 for AML (n=5310), ALL (n=1883), CML (n=1079) and MDS (n=1197)] was interrogated and CMV reactivation was associated with higher non-relapse mortality as well as lower overall survival among all 4 disease categories. Furthermore, in a recent study of 208 patients transplanted between 2008-2013, the average length of in-hospital stay was found to be prolonged by 26 days in patients with CMV reactivation, while the occurrence of more than one CMV reactivation episode increased the costs of an allogeneic HSCT by 25-30% (p<0.0001), highlighting the economic burden of CMV management.

Foscarnet and ganciclovir are frequently used to treat CMV infections after HSCT. However, outside of ganciclovir for CMV retinitis, their use is off-label, and both drugs are associated with significant side effects, particularly renal disease and graft suppression. When used prophylactically, letermovir, a cytomegalovirus DNA terminase complex inhibitor, decreased the incidence of CMV infection/reactivation post-transplant6, and since FDA approval (for CMV prophylaxis in adult HSCT patients) in 2017, is increasingly used in high-risk patients. However the CMV Resistance Working Group of the multidisciplinary CMV Drug Development Forum expects that the wider prophylactic use of letermovir will increase the emergence of organisms that are resistant to conventional antivirals if a CMV breakthrough infection does occur. Indeed, letermovir-resistant CMV strains are increasingly reported and clinical outcomes in those with resistant disease are poor and associated with progressive tissue disease and mortality.

CMVSTs provide an alternative strategy to target both initial reactivations as well as drug-resistant viral strains, as previously reported by our group and others. Indeed 30% of the patients treated with CMVSTs in the current study were infected with viral strains confirmed to be resistant to one or more conventional antiviral drugs.

One goal of the current study was to prepare a CMV-specific T cell bank with sufficient diversity to cover the majority of allogeneic HSCT recipients referred for treatment. Thus, the HLA types of >600 allogeneic HSCT recipients were prospectively compared with a pool of diverse healthy, eligible (CMV seropositive) donors from whom CMVSTs could be generated to identify the minimum cohort that would provide the patients with well-matched products. Using this model it was found that only 8 well-selected donors would provide >95% of the patient population with a T cell product that was matched on at least 2 HLA antigens; further increasing the donor pool would not significantly increase the number of matches. The current study, in which a suitable line was identified for 28 of 29 patients (96.5%) screened for clinical participation, supports the theory that such a donor bank could effectively supply the majority of the allogeneic HSCT patient population.

The racial and ethnic diversity was compared within the transplant patient population with that of the U.S. transplant population (Table 4). This revealed that the diversity within our patient population was similar if not slightly more diverse than the U.S. population. This suggests that the small cell bank developed for the current study could be broadly applied for clinical use across the country. Universal use of the tested CMVSTs across transplant centers is made more feasible by the ability to produce sufficient material to generate cells for >2,000 infusions from a single donor collection. Thus, one could envisage a decentralized distribution model of “off the shelf” third party virus-reactive T cells, ensuring on-demand availability of cells.

In summary, the data indicate that a well characterized bank of CMV-reactive T cells prepared from just 8 well-chosen third party donors can supply the majority of patients with refractory CMV infections with an appropriately matched line that can provide safe and effective antiviral activity.

TABLE 1
Characteristics of generated VST lines.

| | CMV | CMV | | | | | | | | | | | | |
|---|---|---|---|---|---|---|---|---|---|---|---|---|---|---|
| VST | Specificity | Specificity | | | | | CD45RO+/ | CD45RO+/ | | | | | # of | # of |
| line | SFC/1 × 10⁵ | SFC/1 × 10⁵ | CD3 | CD4 | CD8 | CD56 | CD62L+ | CD62L− | HLA- | HLA- | HLA- | HLA- | patients | patients |
| (C#) | IE1 | pp65 | (%) | (%) | (%) | (%) | (%) | (%) | A | B | DR | DQ | Screened* | treated |
| 6790 | 127 | 1186 | 97.81 | 74.23 | 19.48 | 3.88 | 75.45 | 16.33 | 02, 33 | 15, 44 | 07, 13 | 02, 06 | 4 | 3 |
| 6798 | 612 | 805 | 98.79 | 17.75 | 75.73 | 4.05 | 40.3 | 44.83 | 02, 02 | 40, 52 | 04, 08 | 03, 03 | 6 | 4 |
| 6802 | 113 | 1354 | 99.66 | 5.20 | 92.82 | 1.69 | 69.75 | 27.51 | 11, 23 | 35, 57 | 01, 07 | 03, 05 | 1 | 0 |
| 6808 | 827 | 986 | 99.77 | 12.59 | 83.18 | 3.10 | 74.09 | 20.13 | 02, 24 | 40, 52 | 04, 13 | 03, 06 | 4 | 1 |
| 6814 | 639 | 2573 | 99.68 | 28.25 | 69.85 | 0.99 | 41.56 | 55.78 | 2, 24 | 8, 14 | 01, 03 | 02, 05 | 1 | 1 |
| 6823 | 700 | 717 | 99.39 | 10.99 | 86.49 | 1.51 | 47.59 | 48.59 | 11, 68 | 07, 35 | 03, 07 | 02, 02 | 3 | 1 |
| 6834 | 128 | 725 | 99.77 | 15.40 | 82.90 | 2.27 | 64.64 | 32.72 | 02, 24 | 15, 35 | 04, 09 | 03, 03 | 6 | 1 |
| 6838 | 205.5 | 211 | 99.75 | 5.57 | 87.46 | 8.76 | 54.50 | 36.42 | 02, 30 | 13, 35 | 07, 08 | 02, 06 | 1 | 0 |

SFC = spot forming cells;
* = indicates how frequently the VST lines was determined to be the most suitable line for a screened patient.

TABLE 2
Patient characteristics

| Patient | | | | | Type of | R/D CMV | # of | Days post- |
|---|---|---|---|---|---|---|---|---|
| ID# | Age | Ethnicity | Race | Diagnosis | transplant | serostatus | Infusions | transplant |
| 3910 | 12 | Non- | African | Sickle Cell | MRD | Neg/Pos | 1 | 61 |
| | | Hispanic | American | Anemia | | | | |
| 3944 | 45 | Hispanic | White | AML | UCB | Pos/Neg | 1 | 197 |
| 3976 | 13 | Hispanic | White | ALL | MUD | Pos/Pos | 2 | 46 |
| 3762 | 10 | Hispanic | White | HLH | MMUD | Pos/Neg | 1 | 161 |
| 3967 | 51 | Non- | White | AML | UCB | Pos/Neg | 1 | 365 |
| | | Hispanic | | | | | | |
| 4091 | 70 | Non- | White | CTCL | Haplo | Pos/Pos | 1 | 215 |
| | | Hispanic | | | | | | |
| 4115 | 3 | Non- | White | Fanconi | MUD | Pos/Pos | 1 | 105 |
| | | Hispanic | | Anemia | | | | |
| 4170 | 3 | Non- | African | Sickle Cell | MRD | Neg/Pos | 1 | 76 |
| | | Hispanic | American | Anemia | | | | |
| 4134 | 16 | Non- | White | SCID | MUD | Pos/Pos | 1 | 218 |
| | | Hispanic | | | | | | |
| 4201 | 11 | Non- | White | Anaplastic | MUD | Pos/Neg | 3 | 70 |
| | | Hispanic | | Large cell | | | | |
| | | | | lymphoma | | | | |

AML: Acute myeloid leukemia,
ALL: Acute lymphoblastic leukemia,
HLH: Hemophagocytic Lymphohistiocytosis,
CTCL: Cutaneous T-cell lymphoma,
SCID: Severe combined immunodeficiency,
MRD: Matched related donor,
UCB: umbilical cord blood,
MUD: Matched unrelated donor,
MMUD: mismatched unrelated donor,
Haplo: Haploidentical,
R/D: Recipient/Donor,
AKI: Acute kidney injury,
CR: Complete response,
PR: Partial response,
AdV: Adenovirus.

TABLE 3
GvHD pre and post infusion

| | | | GvHD | | |
|---|---|---|---|---|---|
| Patient | Prior | | Rx/PPx at | | |
| ID # | GvHD | Baseline | infusion | aGvHD | cGvHD |
| 3910 | None | None | Cyclosporine | None | None |
| 3944 | None | None | Tacrolimus | None | None |
| 3976 | None | None | Tacrolimus | None | None |
| 3762 | None | None | None | None | None |
| 3967 | GI Grade | None | Sirolimus | None | None |
| | II | | | | |
| 4091 | GI, skin | None | Tacrolimus | None | None |
| | Grade II | | | | |
| 4115 | None | None | None | None | None |
| 4170 | None | None | Tacrolimus | None | None |
| 4134 | GI Grade | None | None | None | None |
| | III | | | | |
| 4201 | None | None | Tacrolimus | None | None |

aGvHD: acute Graft versus Host Disease,
cGvHD: chronic Graft versus Host Disease,
GI: Gastrointestinal,
Rx: Treatment,
PPx: Prophylaxis.

TABLE 4
Racial diversity of allogeneic HSCT recipients.
A total of 174 Program transplant
centers are represented in the US analysis. Each
of these centers performed at least one
unrelated or related donor transplant over the
three-year window of time from Jan. 1,
2013, to Dec. 31, 2015.

| | | Baylor CCGT |
|---|---|---|
| | US (2013-2015) | (2014-2018) |
| Patient Race | Number (%) | Number (%) |
| White | 19,600 (82%) | 608 (74.8%) |
| Black or African American | 2,162 (9%) | 141 (17.3%) |
| Asian | 1,022 (4%) | 49 (6.0%) |
| Pacific Islander | 65 (<1%) | 2 (<1%) |
| American Indian or Alaskan | 133 (1%) | 10 (1.2%) |
| Native | | |
| Multiple Race^{a} | 160 (1%) | n/a |
| Unknown | 704 (3%) | n/a |
| Total | 23,846 | 810 |
| | (100%) | (100%) |

### Example 2. Prophylactic Activity of 3^{rd} Party T Cells: Multivirus-Specific T Lymphocytes for the Prevention of Infections Following Allo-HSCT

In healthy individuals, T cell immunity defends against BKV and other viruses. In allo-HSCT recipients the use of potent immunosuppressive regimens (and subsequent associated immune compromise) leaves patients susceptible to severe viral infections. Therefore, our approach is to restore T cell immunity by the administration of ex vivo expanded, nongenetically modified, virus-specific T cells (VSTs) to control viral infections and eliminate symptoms for the period until the transplant patient's own immune system is restored. To achieve this goal we have prospectively manufactured VSTs from peripheral blood mononuclear cells (PBMCs) procured from healthy, pre-screened, seropositive donors, which are available as a partially HLA-matched “off-the-shelf” product. Viralym-M is one such “off-the-shelf” product.

Viralym-M is specific for five viruses [EBV, CMV, AdV, BKV and Human Herpes virus 6 (HHV6)]. Donor minibanks were constructed as described in Example 1 for making Viralym-M cell lines. Our goal was to generate minibanks with sufficient diversity to cover the majority of allogeneic HSCT recipients referred for treatment.

The Viralym-M manufacturing process was as previously described by the inventors in WO2013/119947 and Tzannou et al., J Clin Oncol. 2017 Nov. 1; 35(31: 3547-3557, each of which is incorporated herein by reference in its entirety and is outlined in FIG. 2. Briefly, PBMCs were isolated from healthy seropositive donors and 250×10⁶ PBMCs were cultured in a G-Rex 100M culture system (Wilson Wolf, Saint Paul, Minn.) in the presence of complete medium, pepmixes covering the Viralym M antigens (adenovirus, CMV, EBV, BKV, and HHV6), IL-4, and IL-7 for around 7-14 days at 37 degrees C. at 5% CO₂ (although the culture time may be increased to around 18 days in some instance). After culturing, Viralym M cell lines were harvested, washed, and aliquoted for cryopreservation in liquid nitrogen until use in quality control testing or as a therapeutic.

Viralym-M was evaluated in a Phase 2 open-label proof-of-concept trial where VSTs were administered to 58 allogeneic HSCT patients with treatment-refractory infections. This trial is referred to herein as CHARMS. The primary objective of CHARMS, which was not statistically powered for superiority or significance, was to determine the feasibility and safety of administering partially HLA-matched multi-VST therapies specific for five viruses in HSCT patients with persistent viral reactivations or infections. Patients were eligible following any type of allogeneic transplant if they had BKV, CMV, AdV, EBV, HHV-6 and/or JCV infections that were relapsed, reactivated or persistent despite standard antiviral therapy.

To assess the alloreactive potential of multivirus-specific T cells (Viralym-M cells) we first directly activated PBMCs with peptide mixtures spanning immunogenic antigens derived from each virus; —Adv (Hexon and Penton), CMV (IE1 and pp65), EBV (LMP2, EBNA1, BZLF1), BK virus (VP1 and large T), and HHV6 (U90, U11 and U14). We then transferred cells to the G-Rex device in T cell medium supplemented with IL4+7 and assessed their cytotoxic activity against HLA-mismatched targets. These cells exhibited minimal/no detectable alloreactivity, supporting the potential safety of these cells when administered as an “off the shelf” partially HLA matched product.

We subsequently explored the safety and clinical effects of partially HLA-matched Viralym-M cells for the treatment of refractory viral infections in children and adults following allogeneic HSCT (Tzannou et al, JCO, 2017). All infusions were well tolerated. Except for 3 patients who developed a transient fever and one who developed lymph node pain within 24 hours of infusion, no acute toxicities were observed. None of the patients developed cytokine release syndrome (CRS). In the ensuing weeks after infusion, one patient developed recurrent Grade III gastrointestinal (GI) GVHD following rapid steroid taper, and eight patients developed recurrent (n=4) or de novo (n=4) Grade I-II skin GVHD, which resolved with the administration of topical treatments (n=7) and re-initiation of corticosteroids after taper (n=1).

For sixty infections in the 52 treated patients who provided evaluable data, the cumulative clinical response rate was 93% by week 6 post Viralym-M infusion, as summarized below:
BKV: Twenty-two patients received Viralym-M for the treatment of persistent viral BKV infection and tissue disease (20 with BK-hemorrhagic cystitis and 2 with BKV-associated nephritis). All 20 BK-HC patients had resolution of clinical symptoms after receiving Viralym-M with 9 complete responses (CRs) and 11 partial responses (PRs), for a 6-week cumulative response of 100%.
CMV: Twenty patients received Viralym-M for persistent CMV. 19 patients responded to Viralym-M with 7 CRs and 12 PRs with 1 non-responder (NR), for a 6-week cumulative response rate of 95%. Responders included 2 of 3 patients with colitis and 1 patient with encephalitis.
AdV: Eleven patients received Viralym-M for persistent AdV and infusions produced 7 CRs, 2 PRs, and 2 NRs, with a 6-week cumulative response rate of 81.8%.
EBV: Three patients received Viralym-M for the treatment of persistent EBV. Two patients achieved a virologic CR and one patient a PR.
HHV6: Four patients received Viralym-M to treat HHV6 reactivations including one patient with refractory encephalitis, and three patients had a PR within 6 weeks of infusion (including the patient with encephalitis) while one did not respond to the treatment.
Dual infections: Eight patients received Viralym-M for two viral infections, with an overall experience of 12 CRs and 4 PRs following a single infusion. CMV, AdV, and EBV were cleared in all cases, all patients with BKV HC had clinical improvement (n=3) or disease resolution (n=2) and the patient with HHV6 encephalitis also had clinical improvement.

We examined the data available from our Phase I/II Viralym-M study to determine whether there was a threshold of HLA matching associated with clinical efficacy. On our clinical trial the products that were used clinically were matched at ⅛ (n=2), 2/8 (n=10), ⅜ (n=11), 4/8 (n=14), ⅝ (n=14), 6/8 (n=4), or ⅞ (n=5) HLA alleles. To determine whether there was a correlation with clinical outcome and degree of HLA matching, we segregated patients into complete response (CR), partial response (PR), and non-responders (NR), but as summarized in FIG. 35, the results suggest that there was no difference in outcome based on the number of HLA matching alleles.

We next examined whether there was a difference in outcome based on the administration of lines matched at HLA class I only, class II only, or a combination of both. Of note, the majority of patients received lines that were matched on both class I and class II alleles and again the results suggest that outcome was not influenced by degree of allele matching.

Moreover, importantly, the CHARMS study demonstrated that it is safe and efficacious to administer more than one different VST product (Viralym M), even if the second line is highly mismatched. For example, as is reported in Tzannou (2017), several patients received administration of two separate cell lines with beneficial responses:

TABLE 5
Selected patient responses (modified from Tzannou (2017)).

| | | | HLA | Best | |
|---|---|---|---|---|---|
| | | | Matching | Response | |
| Patient | | Lines | (of eight | by 6 | |
| No. | Infection | Infused | lines) | Weeks | Outcome |
| 3848 | resistant | C5404; | 3 alleles; | PR; no | PR with |
| | strain | C5678 | 4 alleles | | recurrence |
| | CMV | | | | at 4 |
| | | | | | weeks |
| 3357 | CMV | C5678; | 4 alleles; | PR | Sustained CR |
| | | C6323 | 5 alleles | | |
| 4076 | CMV, | C6209; | 6 alleles; | CMV CR; | Sustained CR |
| | AdV | C6611 | 3 alleles | AdV CR | for CMV; |
| | | | | | recurrence of |
| | | | | | AdV with |
| | | | | | sustained |
| | | | | | CR after |
| | | | | | second |
| | | | | | infusion |
| 3755 | EBV, | C5602, | 5 alleles; | EBV CR; | Sustained CR |
| | BKV | C5624 | 2 alleles | BKV PR | for EBV; |
| | | | | | PR for BKV |
| | | | | | with stable |
| | | | | | renal function |
| 3877 | BKV | C6322, | 3/6 alleles; | Virologic | Resolution |
| | | C5602 | 4 alleles | PR; | of HC after |
| | | | | Clinical | third infusion |
| | | | | PR | |
| 3899 | BKV | C6726, | 4 alleles; | Virologic | Resolution |
| | | C5497 | 3 alleles | PR; | of HC after |
| | | | | Clinical | second |
| | | | | PR | infusion |

Moreover, as shown below in Table 6 (modified from Tzannou (2017)), these patients that received administration of at least two cell lines showed no or little GVHD by week 6 or cGVHD within 1 year of treatment.

TABLE 6
Selected patient responses

| Patient | | aGVHD by Week 6 | cGVHD Within 1 Year |
|---|---|---|---|
| No. | Infection | (treatment; outcome) | (treatment; outcome) |
| 3848 | resistant | NO | N/ANO |
| | strain CMV | | |
| 3357 | CMV | Grade 1 skin | NO |
| | | (topical | |
| | | corticosteroids; | |
| | | resolved) | |
| 4076 | CMV, AdV | NO | NO |
| 3755 | EBV, BKV | NO | quiescent |
| | | | chronic GVHD |
| 3877 | BKV | Grade 1 skin (topical | NO |
| | | corticosteroids; | |
| | | resolved) | |
| 3899 | BKV | NO | N/A |

Abbreviations: GVHD: graft versus host disease;
aGVHD: acute GVHD;
cGVHD: chronic GVHD;
N/A: not applicable.

Thus, these results from this Phase I/II data demonstrated that >95% of patients received a product matching at ≥2 HLA alleles, which was associated with clinical benefit. Matching on HLA class I or class II did not appear to influence outcome and did not impact the safety profile of the cells, nor did administering more than one cell line to a given patient, even when second line was highly mismatched.

The data were then examined for evidence that the 3^{rd} party T cells have prophylactic potential.

First, persistence of 3^{rd} part VSTs with specificity against a virus for which the patients did not reactivate was confirmed in a total of 4 patients. For example, one patient (HLA matched at 2 alleles with the VST line used for treatment; see Table 7) was treated for BK HC. The VST line infused had BKV and CMV activity mediated in the context of HLA-A2 (shared allele). Persistence of the VST was tracked by analyzing immune responses presented in the context of DR3 (unique to the VST line). Endogenous immune reconstitution was monitored by tracking immune responses to peptides presented by B40 and DR13 (alleles unique to the patient).

TABLE 7
Patient and 3^{rd} party VST cell line alleles

| Patient | A2, 3 | B40 | C3 | DR13 | DQ6 |
|---|---|---|---|---|---|
| VST Line | A2 | B8, 15 | C3, 7 | DR3, 4 | DQ2, 3 |

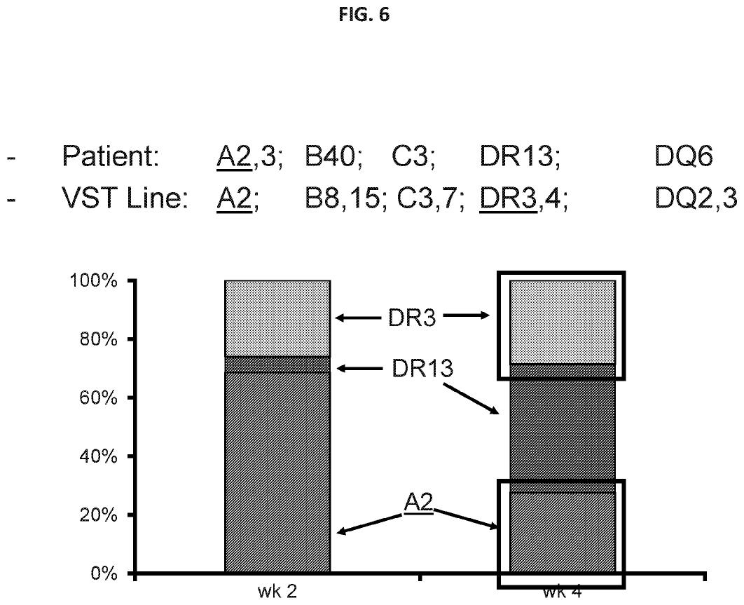

Even at 4 weeks post infusion, peptide-specific immune responses against peptides presented in the context of HLA-A2, which were CMV-specific, could be detected. (FIG. 6). CMV-specific responses in the context of DR3 could also be detected, indicating that the 3^{rd} party VSTs were present (FIG. 6). Thus, the study showed that surprisingly, the infusion of the 3^{rd} party VSTs in a patient being treated for BK HC provided prophylactic CMV coverage that prevented a CMV reactivation. A similar pattern was observed in additional patients as described in detail below, confirming persistence of the VSTs in vivo out to 12 weeks post-infusion.

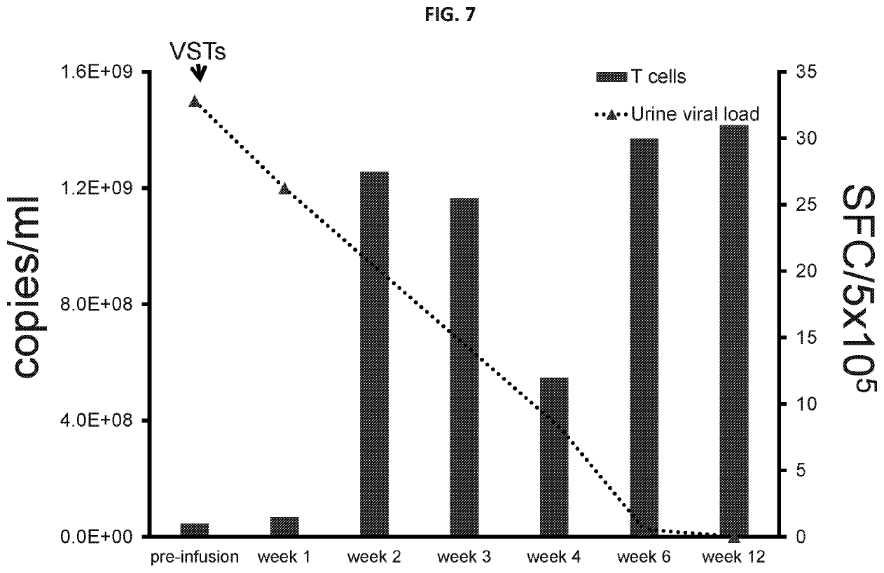

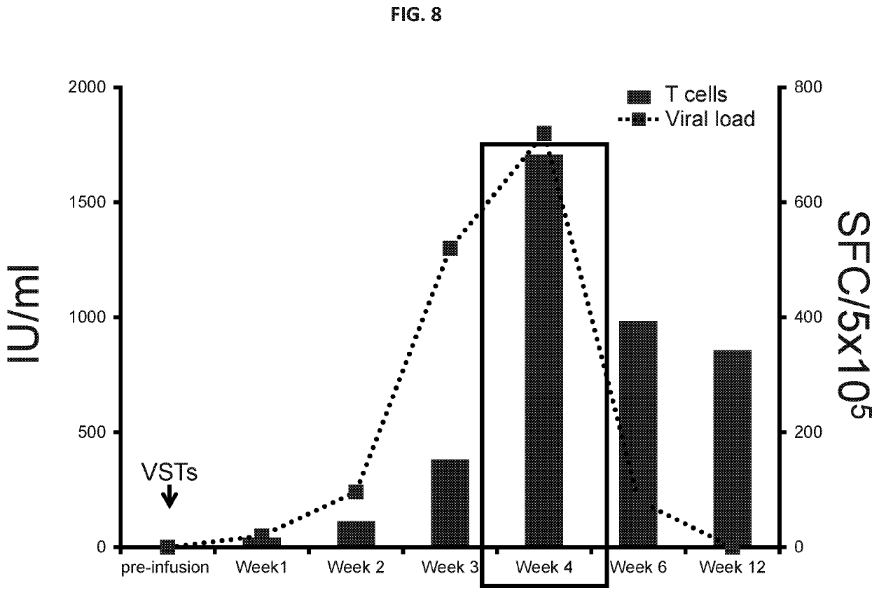

Moreover, 3^{rd} party VSTs were detected in a patient who experienced a viral reactivation post-infusion. The patient received VST to treat BKV HC. Subsequently, the patient reactivated CMV. FIG. 7 shows the patient's BK response. A typical profile with a decrease in viral load corresponding to BKV-specific T cell expansion after infusion was observed (FIG. 7). The CMV reactivation occurred two weeks after the VSTs were infused. The viral load and T cell expansion are shown in FIG. 8. As soon as the virus reactivated at 2 weeks post VST infusion, the CMV-specific T cells responded, controlling the virus without other medication. At the Week 4 timepoint, the presence of the 3^{rd} party VSTs was confirmed using the persistence analysis discussed above. CMV-specific cells remained until at least week 12, and the CMV viral load was undetectable by week 12.

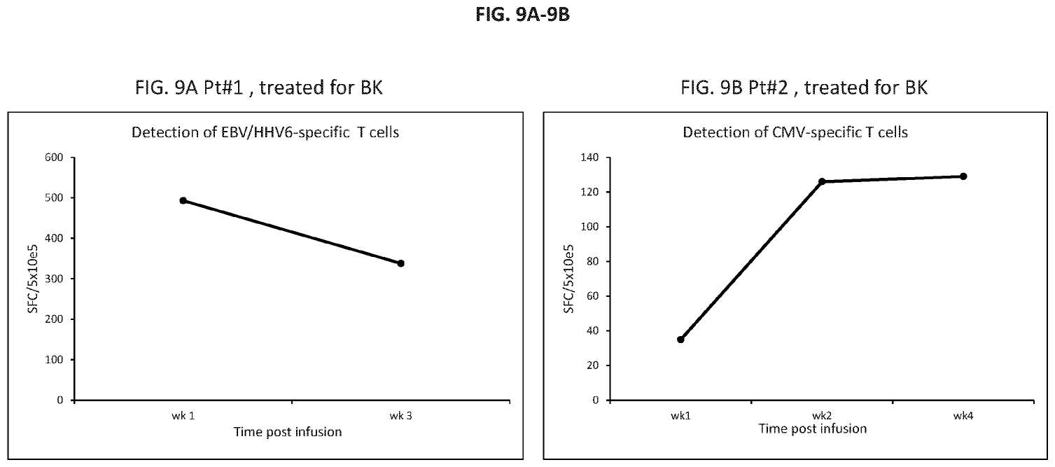

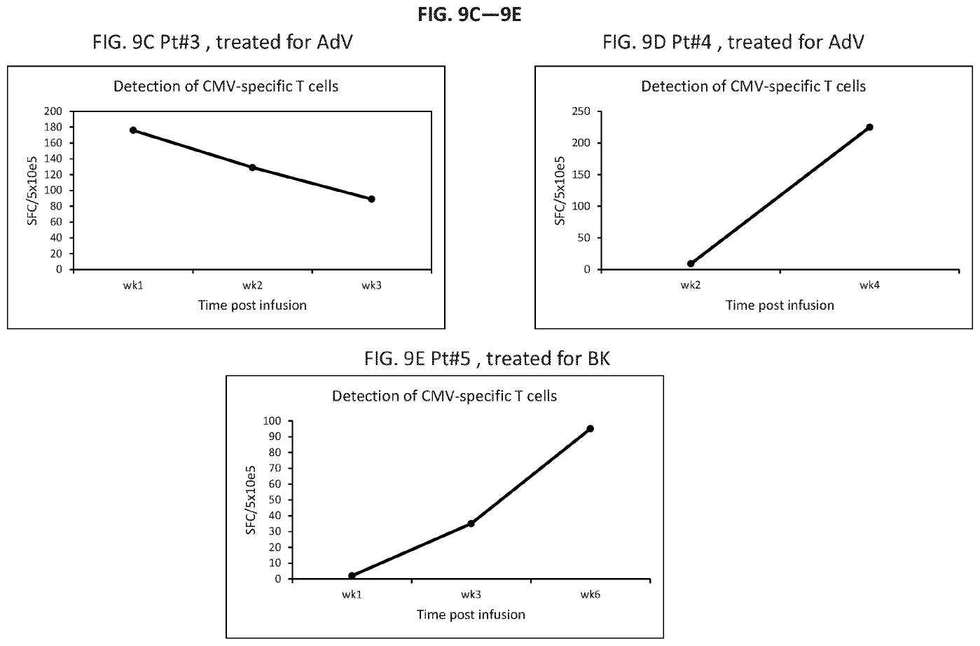

Additional evidence that Viralym-M-derived T cells persisted in recipients is provided in FIGS. 9A-9E. For example, in one patient treated for a BK infection, Viralym-M-derived HHV6 and EBV specific T cells were detectable out to at least 3 weeks post-infusion (the last timepoint tested). The peptide reactivities detected in this patient were an EBV-LMP2 HLA-A 1-restricted response and an HHV6-U90 HLA-A1-restricted response. These specificities were unique to the line infused, and HLA-A1 was not expressed by the patient. Thus, the detected activity was derived from the infused VST line which persisted for at least 3 weeks (FIG. 9A). In a second patient treated for a BK infection, Viralym-M-derived CMV specific T cells were detected out to 4 weeks post-infusion (the last timepoint tested). The peptide reactivities detected in this patient were a CMV-IE1 HLA-B8-restricted response and a CMV-pp65 HLA-DR4-restricted response, both of which were unique to the line infused, and HLA-B8 or DR4 were not expressed by the patient; thus confirming that the detected activity was derived from the infused VST line (FIG. 9B). In a patient treated for an AdV infection, Viralym-M-derived CMV specific T cells were detected out to 3 weeks post 2nd infusion (the last timepoint tested). The peptide reactivity detected was a CMV-pp65 HLA-DR4-restricted response which was unique to the line infused, and HLA-DR4 was not expressed by the patient. Thus, the detected activity was derived from the infused VST line (FIG. 9C). In another patient treated for an AdV infection, Viralym-M-derived CMV specific T cells were detected out to 4 weeks post infusion (the last timepoint tested;). The peptide reactivity detected was a CMV-IE1 HLA-B8-restricted response which was unique to the line infused, and HLA-H8 was not expressed by the patient. Thus, the detected activity in this patient was derived from the infused VST line (FIG. 9D). Finally, in another patient who was treated for a BKV infection, Viralym-M-derived CMV specific T cells were detected out to 12 weeks post infusion (the last timepoint tested). The peptide reactivities detected were CMV-pp65 HLA-DR4- and DR15-restricted responses, both of which were unique to the line infused, and HLA-DR4 or DR15 were not expressed by the patient. Thus, the activity was detectable for at least 12 weeks and was derived from the infused VST line (FIG. 9E).

Table 8 provides a summary of evidence of prophylactic protection with Viralym-M. In patients treated with Viralym-M for active BKV or AdV, CMV VSTs were detected for at least 3 weeks, 4 weeks, or 12 weeks as indicated. In the patient discussed above and in FIG. 7, elimination of reactivated CMV without further treatment was achieved and CMV specific VSTs were detected at least 12 weeks after administration of Viralym-M. In the four other patients assessed, no reactivation of the virus against which 3^{rd} party T cells were detected occurred.

TABLE 8
Persistence in recipients and prophylactic protection of 3^{rd} party VSTs.

| | Reason for | | 3^{rd} party | Duration | |
|---|---|---|---|---|---|
| | treatment with | Subsequent | T cells | of VST | |
| | Viralym-M | reactivation | detected | detection* | Outcome |
| 1 | BKV | CMV | CMV | 12 weeks | CMV control |
| | | | | | without |
| | | | | | further |
| | | | | | treatment |
| 2 | BKV | None | CMV | 4 weeks | No reactivation |
| 3 | AdV | None | CMV | 4 weeks | No reactivation |
| 4 | BKV | None | HHV6, | 3 weeks | No reactivation |
| | | | EBV | | |
| 5 | AdV | None | CMV | 3 weeks | No reactivation |
| | | | | (post | |
| | | | | second | |
| | | | | infusion) | |

*Duration of VST detection indicates last timepoint tested in each suject. Thus, the VSTs persisted for at least the indicated duration for each patient, and may rave persisted for longer.

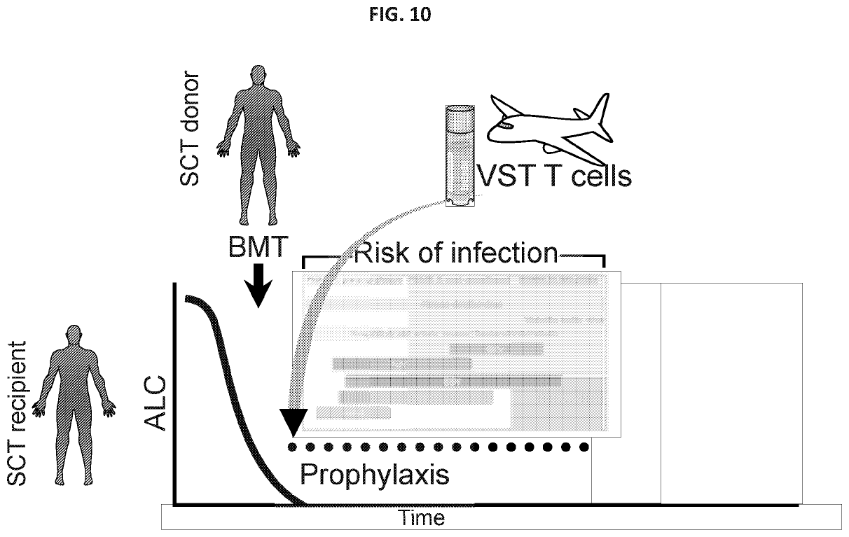

Therefore, strikingly, the infusion of 3^{rd} party VSTs provides prophylactic protection against viruses not yet present or not yet reactivated in addition to targeting an active infection. Thus, the 3^{rd} party VST compositions and methods can be used to prophylactically provide broad spectrum protection in vivo. This is a particularly important clinical advantage for patients who are immunocompromised for any reason. A schematic of the prophylactic method is provided as FIG. 10.

### Example 3. Clinical Studies Addressing Prophylactic Therapy of 3^{rd} Party VSTs

A Phase II, double-blind, placebo controlled trial of Viralym-M for the prevention of clinically significant viral infections (AdV, BKV, CMV, EBV, and HHV6) in high risk patients following allogeneic HSCT is conducted. Study objectives include persistence of functional Viralym-M T cells; reduction in AdV, BKV, CMV, EBV and/or HHV6 infections requiring treatment or reduction in disease development; success of donor engraftment; all-cause and non-relapse mortality at 1-year post-transplant. Inclusion criteria include recipients of allo-HSCTs of any age who are at high-risk for clinically significant viral infections (e.g., defined as patients who received myeloablative allo-HSCT using either bone marrow, single/double cord blood or peripheral blood stem cells from an unrelated donor or a haploidentical donor; those receiving T cell depleted grafts or those receiving post-transplant cyclophosphamide), who are seropositive for AdV, BKV, CMV, EBV and/or HHV6. Patients must be asymptomatic at the time of screening. Exclusion criteria: ongoing therapy with corticosteroids (prednisone dose >0.5 mg/kg/day or equivalent); prior therapy with anti-thymocyte globulin (ATG), alemtuzumab (Campath-1H) or other immunosuppressive T cell monoclonal antibodies within 28 days of screening for enrollment; received donor lymphocyte infusion (DLI) or CD34+ stem cell top-up within 28 days of screening for enrollment; evidence of Grade >2 acute GVHD; presence of other progressing infections (can be viral, fungal or bacterial in origin; progressing infection is defined as hemodynamic instability attributable to sepsis or new symptoms, worsening physical signs or radiographic findings attributable to infection); presence of encephalitis; requirement for FiO2>0.5 to maintain arterial oxygen saturation >90%; hemoglobin <8 gm/dL despite RBC transfusions; renal dysfunction defined as estimated glomerular filtration rate (GFR)<30 ml/min/1.73 m2; females who are pregnant or breastfeeding and relapse of primary malignancy.

Patients will be consented and screened pre-transplant. If patients meet eligibility criteria, they will be enrolled and randomized. After randomization, patients will receive an infusion of a fixed cell dose of 2×10⁷ Viralym-M cells (body weight <40 kg) or 4×10⁷ Viralym-M cells (body weight >40 kg) (or) placebo 28 days post-transplant (provided they meet eligibility criteria at the time of infusion). Patients will be monitored for viremia and viruria, and/or monitored for the persistence of virus specific 3^{rd} party VSTs. Some subjects may receive multiple infusions of the same and/or different 3^{rd} party VSTs. For example, patients may be administered a first dose of 3^{rd} party VSTs followed by a second dose about 6, about 8, about 10, or about 12 weeks later. Some subjects may continue to receive 3^{rd} party VSTs about every 6 weeks, about every 8 weeks, about every 10 weeks, or about every 12 weeks for the duration of the study and/or until the patient is no longer immunocompromised. The study will show that AdV, BKV, CMV, EBV, and/or HHV6 infection can be prevented in immunocompromised patients via the administration of 3^{rd} party VSTs, even where the 3^{rd} party VSTs are administered prior to any infection with or reactivation of the virus.

Similar studies are carried out to assess the prophylactic therapy with 3^{rd} party VSTs specific for HHV8, HBV, or SARS-CoV-2. Patients who do not have detectable virus are administered 3^{rd} party VSTs specific for HHV8, HBV, or SARS-CoV-2 and monitored for viral load and/or for the persistence of the virus-specific 3^{rd} party VSTs in the recipient. Some subjects may receive multiple infusions of the same and/or different 3^{rd} party VSTs specific for the indicated virus. For example, patients may be administered a first dose of 3^{rd} party VSTs specific for HHV8, HBV, or SARS-CoV-2, followed by a second dose about 6, about 8, about 10, or about 12 weeks later. Some subjects may continue to receive 3^{rd} party VSTs about every 6 weeks, about every 8 weeks, about every 10 weeks, or about every 12 weeks for the duration of the study and/or until the patient is no longer immunocompromised. The study will show that HHV8, HBV, and SARS-CoV-2 can each be prevented and/or readily controlled in immunocompromised patients via the administration of 3^{rd} party VSTs, even where the 3^{rd} party VSTs are administered prior to any infection with or reactivation of the virus.

Similar studies are carried out to assess the prophylactic therapy with 3^{rd} party VSTs specific for RSV, influenza, PIV, and hMPV. Patients who do not have detectable virus are administered 3^{rd} party VSTs specific for RSV, influenza, PIV, and hMPV and monitored for viral load and/or for the persistence of the virus-specific 3^{rd} party VSTs in the recipient. Some subjects may receive multiple infusions of the same and/or different 3^{rd} party VSTs specific for the indicated virus. For example, patients may be administered a first dose of 3^{rd} party VSTs specific for RSV, influenza, PIV, and hMPV, followed by a second dose about 6, about 8, about 10, or about 12 weeks later. Some subjects may continue to receive 3^{rd} party VSTs about every 6 weeks, about every 8 weeks, about every 10 weeks, or about every 12 weeks for the duration of the study and/or until the patient is no longer immunocompromised. For example, patients may be administered a first dose of 3^{rd} party VSTs specific for RSV, influenza, PIV, and hMPV, followed by a second dose 6, 8, 10, or 12 weeks later. Some subjects may continue to receive 3^{rd} party VSTs every 6 weeks, every 8 weeks, every 10 weeks, or every 12 weeks for the duration of the study and/or until the patient is no longer immunocompromised. The study will show that RSV, influenza, PIV, and hMPV can each be prevented and/or readily controlled in immunocompromised patients via the administration of 3^{rd} party VSTs, even where the 3^{rd} party VSTs are administered prior to any infection with or reactivation of the virus.

### REFERENCES

1. Teira P, Battiwalla M, Ramanathan M, Barrett A J, Ahn K W, Chen M, et al. Early cytomegalovirus reactivation remains associated with increased transplant-related mortality in the current era: a CIBMTR analysis. Blood. 2016; 127(20):2427-38.
2. Chan S T, Logan A C. The clinical impact of cytomegalovirus infection following allogeneic hematopoietic cell transplantation: Why the quest for meaningful prophylaxis still matters. Blood reviews. 2017; 31(3):173-83.
3. Blyth E, Withers B, Clancy L, Gottlieb D. CMV-specific immune reconstitution following allogeneic stem cell transplantation. Virulence. 2016; 7(8):967-80.
4. Torma N, Solano C, Benet I, Clari M A, Nieto J, de la Camara R, et al. Lack of prompt expansion of cytomegalovirus pp65 and IE-1-specific IFNgamma CD8+ and CD4+ T cells is associated with rising levels of pp65 antigenemia and DNAemia during pre-emptive therapy in allogeneic hematopoietic stem cell transplant recipients. Bone marrow transplantation. 2010; 45(3):543-9.
5. Walter E A, Greenberg P D, Gilbert M I, Finch R J, Watanabe K S, Thomas E D, et al. Reconstitution of cellular immunity against cytomegalovirus in recipients of allogeneic bone marrow by transfer of T-cell clones from the donor. NEnglJMed. 1995; 333(16):1038-44.
6. Einsele H, Roosnek E, Rufer N, Sinzger C, Riegler S, Loffler J, et al. Infusion of cytomegalovirus (CMV)-specific T cells for the treatment of CMV infection not responding to antiviral chemotherapy. Blood. 2002; 99(11):3916-22.
7. Leen A M, Myers G D, Sili U, Huls M H, Weiss H, Leung K S, et al. Monoculture-derived T lymphocytes specific for multiple viruses expand and produce clinically relevant effects in immunocompromised individuals. NatMed. 2006; 12(10):1160-6.
8. Micklethwaite K, Hansen A, Foster A, Snape E, Antonenas V, Sartor M, et al. Ex vivo expansion and prophylactic infusion of CMV-pp65 peptide-specific cytotoxic T-lymphocytes following allogeneic hematopoietic stem cell transplantation. Biology of blood and marrow transplantation: journal of the American Society for Blood and Marrow Transplantation. 2007; 13(6):707-14.
9. Peggs K S, Verfuerth S, Pizzey A, Chow S L, Thomson K, Mackinnon S. Cytomegalovirus-specific T cell immunotherapy promotes restoration of durable functional antiviral immunity following allogeneic stem cell transplantation. ClinlnfectDis. 2009; 49(12):1851-60.
10. Scheinberg P, Melenhorst J J, Brenchley J M, Hill B J, Hensel N F, Chattopadhyay P K, et al. The transfer of adaptive immunity to CMV during hematopoietic stem cell transplantation is dependent on the specificity and phenotype of CMV-specific T cells in the donor. Blood. 2009; 114(24):5071-80.
11. Peggs K S, Thomson K, Samuel E, Dyer G, Armoogum J, Chakraverty R, et al. Directly selected cytomegalovirus-reactive donor T cells confer rapid and safe systemic reconstitution of virus-specific immunity following stem cell transplantation. ClinlnfectDis. 2011; 52(1):49-57.
12. Blyth E, Clancy L, Simms R, Ma C K, Burgess J, Deo S, et al. Donor-derived CMV-specific T cells reduce the requirement for CMV-directed pharmacotherapy after allogeneic stem cell transplantation. Blood. 2013; 121(18):3745-58.
13. Gerdemann U, Katari U L, Papadopoulou A, Keirnan J M, Craddock J A, Liu H, et al. Safety and clinical efficacy of rapidly-generated trivirus-directed T cells as treatment for adenovirus, EBY, and CMY infections after allogeneic hematopoietic stem cell transplant. MolTher. 2013; 21(11):2113-21.
14. Papadopoulou A, Gerdemann U, Katari U L, Tzannou I, Liu H, Martinez C, et al. Activity of broad-spectrum T cells as treatment for AdY, EBY, CMY, BKY, and HHY6 infections after HSCT. SciTranslMed. 2014; 6(242):242ra83.
15. Schmitt A, Tonn T, Busch D H, Grigoleit G U, Einsele H, Odendahl M, et al. Adoptive transfer and selective reconstitution of streptamer-selected cytomegalovirus-specific CD8+ T cells leads to virus clearance in patients after allogeneic peripheral blood stem cell transplantation. Transfusion. 2011; 51(3):591-9.
16. Feucht J, Opherk K, Lang P, Kayser S, Hartl L, Bethge W, et al. Adoptive T-cell therapy with hexon-specific Th1 cells as a treatment of refractory adenovirus infection after HSCT. Blood. 2015; 125(12):1986-94.
17. Mehta J, Gordon L I, Tallman M S, Winter J N, Evens A M, Frankfurt O, et al. Does younger donor age affect the outcome of reduced-intensity allogeneic hematopoietic stem cell transplantation for hematologic malignancies beneficially? Bone marrow transplantation. 2006; 38(2):95-100.
18. Hague T, Wilkie G M, Jones M M, Higgins C D, Urquhart G, Wingate P, et al. Allogeneic cytotoxic T-cell therapy for EBY-positive posttransplantation lymphoproliferative disease: results of a phase 2 multicenter clinical trial. Blood. 2007; 110(4):1123-31.
19. Uhlin M, Okas M, Gertow J, Uzunel M, Brismar T B, Mattsson J. A novel haplo-identical adoptive CTL therapy as a treatment for EBY-associated lymphoma after stem cell transplantation. Cancer Immunol Immunother. 2010; 59(3):473-7.
20. Barker J N, Doubrovina E, Sauter C, Jaroscak J J, Perales M A, Doubrovin M, et al. Successful treatment of EBY-associated posttransplantation lymphoma after cord blood transplantation usmg third-party EBY-specific cytotoxic T lymphocytes. Blood. 2010; 116(23):5045-9.
21. Qasim W, Derniame S, Gilmour K, Chiesa R, Weber M, Adams S, et al. Third-party virus-specific T cells eradicate adenoviraemia but trigger bystander graft-versus-host disease. British journal of haematology. 2011; 154(I): 150-3.
22. Uhlin M, Gertow J, Uzunel M, Okas M, Berglund S, Watz E, et al. Rapid salvage treatment with virus-specific T cells for therapy-resistant disease. ClinlnfectDis. 2012; 55(8):1064-73.
23. Doubrovina E, Oflaz-Sozmen B, Prockop S E, Kernan N A, Abramson S, Teruya-Feldstein J, et al. Adoptive immunotherapy with unselected or EBY-specific T cells for biopsy-proven EBV+ lymphomas after allogeneic hematopoietic cell transplantation. Blood. 2012; 119(11):2644-56.
24. Leen A M, Bollard C M, Mendizabal A M, Shpall E J, Szabolcs P, Antin J H, et al. Multicenter study of banked third-party virus-specific T cells to treat severe viral infections after hematopoietic stem cell transplantation. Blood. 2013; 121(26):5113-23.
25. Vickers M A, Wilkie G M, Robinson N, Rivera N, Hague T, Crawford D H, et al. Establishment and operation of a Good Manufacturing Practice-compliant allogeneic Epstein-Barr virus (EBV)-specific cytotoxic cell bank for the treatment of EBY-associated lymphoproliferative disease. BrJHaematol. 2014; 167(3):402-10.
26. Gallot G, Vollant S, Saiagh S, Clemenceau B, Vivien R, Cerato E, et al. T-cell therapy using a bank of EBY-specific cytotoxic T cells: lessons from a phase 1/11 feasibility and safety study. J Immunother. 2014; 37(3): 170-9.
27. Tzannou I, Papadopoulou A, Naik S, Leung K, Martinez C A, Ramos C A, et al. Off-the-Shelf Virus-Specific T Cells to Treat B K Virus, Human Herpesvirus 6, Cytomegalovirus, Epstein-Barr Virus, and Adenovirus Infections After Allogeneic Hematopoietic Stem-Cell Transplantation. J Clin Oneal. 2017; 35(31):3547-57.
28. Withers B, Blyth E, Clancy L E, Yong A, Fraser C, Burgess J, et al. Long-term control of recurrent or refractory viral infections after allogeneic HSCT with third-party virus-specific T cells. Blood Adv. 2017; 1(24):2193-205.
29. Filipovich A H, WeisdorfD, Pavletic S, Socie G, Wingard J R, Lee S J, et al. National Institutes of Health consensus development project on criteria for clinical trials in chronic graft-versus-host disease: I. Diagnosis and staging working group report. Biology of blood and marrow transplantation: journal of the American Society for Blood and Marrow Transplantation. 2005; 11(12):945-56.
30. Przepiorka D, Weisdorf D, Martin P, Klingemann H G, Beatty P, Hows J, et al. 1994 Consensus Conference on Acute GVHD Grading. Bone marrow transplantation. 1995; 15(6):825-8.
32. Gerdemann et al. Rapidly Generated Multivirus-Speicfic Cytotoxic T Lymphocytes for the Prophylaxis and Treatment of Viral Infections. Molecular Therapy Vol. 2012; 20(8) 1622-32.
33. Cutler C, Henry N L, Magee C, Li S, Kim H T, Alyea E, et al. Sirolimus and thrombotic microangiopathy after allogeneic hematopoietic stem cell transplantation. Biology of blood and marrow transplantation: Journal of the American Society for Blood and Marrow Transplantation. 2005; 11(7):551-7. doi: 10.1016/j.bbmt.2005.04.007.
34. Liu et al. CRISPR-Cas9-mediated multiplex gene editing in CAR-T cells. Cell Research (2017) 27:154-157.
35. Kagoya et al. Genetic Ablation of HLA Class I, Class II, and the T Cell Receptor Enables Allogeneic T cells to be Used for Adoptive T Cell Therapy. Cancer Immunology Research 8; 926-36 (2020).

1. A method of preventing a viral infection or the reactivation of a latent virus via a third-party allogeneic T cell therapy, the method comprising prophylactically administering to a patient a first antigen-specific T cell line that is a polyclonal third party T cell line, said T cell line comprising antigen specificity for one or more viral antigen and said T cell line comprising an HLA type that matches the patient's HLA type on 2 or more HLA alleles.

2. A method of controlling a viral infection or the reactivation of a latent virus via a third-party allogeneic T cell therapy, the method comprising prophylactically administering to a patient a first antigen-specific T cell line that is a polyclonal third party T cell line, said T cell line comprising antigen specificity for one or more viral antigen and said T cell line comprising an HLA type that matches the patient's HLA type on 2 or more HLA alleles.

3. The method of claim 1 or claim 2, wherein the patient is at a higher risk than an average person in the general population of contracting a viral infection or of having a latent virus reactivate.

4. The method of any one of claims 1-3, wherein the viral infection poses a greater risk to the patient's health or life than such an infection would pose to an average person in the general population.

5. The method of any one of claims 1-4, wherein the patient does not show evidence of an active viral infection or of reactivation of the latent virus when the T cell line is administered.

6. The method of any one of claims 1-5, wherein the patient has no detectable viremia or viruria when the T cell line is administered.

7. The method of any one of claims 1-6, wherein the patient has an absolute lymphocyte count of less than 800 lymphocytes per μL blood.

8. The method of any one of claims 1-7, wherein the patient lacks endogenous T cells.

9. The method of any one of claims 1-8, wherein the patient is seropositive for any one or more of AdV, BKV, CMV, EBV, HHV6, HHV8, RSV, influenza, PIV, hMPV HBV, and SARS-CoV-2.

10. The method of any one of claims 1-9, wherein the first antigen-specific T cell line is administered to the patient a plurality of times.

11. The method of any one of claims 1-10, wherein the first antigen-specific T cell line is administered to the patient in a second administration about 4-12 weeks after a first administration.

12. The method of any one of claims 1-11, wherein the first antigen-specific T cell line is administered to the patient about every 4-12 weeks.

13. The method of claim 12, wherein the patient is immunocompromised, and wherein the first antigen-specific T cell line is administered to the patient about every 4-12 weeks until the patient is no longer immunocompromised.

14. The method of any one of claims 1-13, wherein the patient is administered a composition comprising a peptide or whole antigen that corresponds to the antigen for which the first antigen-specific T cell line is specific, and wherein the peptide or whole antigen is administered to the subject about 4-12 weeks after administration of the first antigen-specific T cell line.

15. The method of claim 14, wherein the composition further comprises an adjuvant.

16. The method of any one of claims 1-15,
(a) further comprising administering to the patient one or more second antigen-specific T cell lines; or
(b) further comprising administering to the patient 2, 3, 4, 5, 6, 7, 8, 9, or 10 more second antigen-specific T cell lines.

17. The method of claim 16, wherein the first and the second antigen-specific T cell lines are administered to the patient concurrently.

18. The method of claim 16, wherein the first and the second antigen-specific T cell lines are administered to the patient sequentially.

19. The method of any one of claims 16-18, wherein the one or more second antigen-specific T cell lines are administered to the patient a plurality of times.

20. The method of claim 19, wherein the patient is immunocompromised, and wherein the one or more second antigen-specific T cell lines are administered to the patient about every 6-12 weeks until the patient is no longer immunocompromised.

21. The method of any one of claims 16-20, wherein at least one, and optionally each, second antigen-specific T cell line comprises the same antigen specificity as the first antigen-specific T cell line, but is generated from a different donor.

22. The method of any one of claims 1-21, wherein the 2 or more HLA alleles that are matched between the patient and the first antigen-specific T cell line and/or any second antigen-specific T cell line if one was administered comprises at least 2 HLA Class I alleles; at least 2 HLA Class II alleles; or at least 1 HLA Class I allele and at least 1 HLA Class II allele.

23. The method of any one of claims 1-22, wherein the viral infection is from a virus selected from EBV, CMV, Adenovirus, BK, JC virus, HHV6, RSV, Influenza, Parainfluenza, Bocavirus, Coronavirus, LCMV, Mumps, Measles, human Metapneumovirus, Parvovirus B, Rotavirus, merkel cell virus, herpes simplex virus, HPV, HIV, HTLV1, HHV8, HBV, West Nile Virus, Zika virus, and Ebola virus.

24. The method of any one of claims 1-23, wherein the first and/or second antigen-specific T cell line comprises antigen specificity for at least one antigen or a portion thereof from a single virus.

25. The method of claim 24, wherein the single virus is selected from EBV, CMV, Adenovirus, BK, JC virus, HHV6, RSV, Influenza, Parainfluenza, Bocavirus, Coronavirus, LCMV, Mumps, Measles, human Metapneumovirus, Parvovirus B, Rotavirus, merkel cell virus, herpes simplex virus, HPV, HIV, HTLV1, HHV8, HBV, West Nile Virus, Zika virus, and Ebola virus.

26. The method of claim 25, wherein the single virus is HBV or HHV8.

27. The method of any one of claims 24-26, wherein the first antigen-specific T cell line comprises specificity for two or more antigens or a portion thereof from the single virus.

28. The method of any one of claims 1-23, wherein the first antigen-specific T cell line comprises antigen specificity for at least one antigen or a portion thereof, from at least two different viruses.

29. The method of any one of claims 1-23, wherein the first antigen-specific T cell line comprises antigen specificity for at least one antigen or a portion thereof, from 1-10 different viruses.

30. The method of any one of claims 1-23, wherein the first antigen-specific T cell line comprises antigen specificity for 2-5 antigens from each of at least two different viruses or at least a portion of 2-5 antigens from each of at least two different viruses.

31. The method of any one of claims 13-30, wherein the second antigen-specific T cell line comprises antigen specificity for at least one antigen or a portion thereof, from 1-10 different viruses.

32. The method of any one of claims 13-31, wherein the second antigen-specific T cell line comprises antigen specificity for 2-5 antigens from each of at least two different viruses or at least a portion of 2-5 antigens from each of at least two different viruses.

33. The method of any one of claims 1-32, wherein the antigen is a viral antigen from a virus selected from EBV, CMV, Adenovirus, BK, JC virus, HHV6, RSV, Influenza, Parainfluenza, Bocavirus, Coronavirus, LCMV, Mumps, Measles, human Metapneumovirus (HMPV), Parvovirus B, Rotavirus, merkel cell virus, herpes simplex virus, HPV, HIV, HTLV1, HHV8, HBV, West Nile Virus, Zika virus, and Ebola virus.

34. The method of any one of claims 1-23, wherein the first and/or the second antigen-specific T cell comprises specificity for at least one antigen from each of the following viruses: RSV, Influenza, Parainfluenza, and HMPV.

35. The method of claim 34, wherein the Influenza antigens are selected from influenza A antigens NP1, MP1, and a combination thereof; the RSV antigens are selected from N, F, and a combination thereof; the hMPV antigens are selected from F, N, M2-1, M, and a combination thereof; and the PIV antigens are selected from M, HN, N, F, and a combination thereof.

36. The method of any one of claims 1-23, wherein the first and/or the second antigen-specific T cell comprises specificity for at least one antigen from each of the following viruses: EBV, CMV, adenovirus, BK, HHV6.

37. The method of claim 36, wherein the EBV antigens are selected from LMP2, EBNA1, BZLF1, and a combination thereof; the CMV antigens are selected from IE1, pp65, and a combination thereof; the adenovirus antigens are selected from Hexon, Penton, and a combination thereof; the BK virus antigens are selected from VP1, large T, and a combination thereof; and the HHV6 antigens are selected from U90, U11, U14, and a combination thereof.

38. The method of any one of claims 1-33, wherein the first and/or the second antigen-specific T cell comprises specificity for at least one antigen from HBV.

39. The method of any one of claims 1-33, wherein the first and/or the second antigen-specific T cell comprises specificity for at least one antigen from HHV8.

40. The method of any one of the preceding claims, wherein the antigen-specific T cells are produced by culturing, in the presence of the antigens or a portion thereof, mononuclear cells from a suitable donor having an HLA type that matches the patient's HLA type on 2 or more HLA alleles.

41. The method of any one of the preceding claims, wherein the antigen-specific T cells are produced by culturing, in the presence of pepmixes spanning the antigens, or a portion thereof, mononuclear cells from a suitable donor having an HLA type that matches the patient's HLA type on 2 or more HLA alleles.

42. The method of claim 40 or 41, wherein the culturing is in the presence of IL4 and IL7.

43. The method of claim 42, wherein the pepmix comprises 15 mer peptides.

44. The method of any one of claims 41-43, wherein the peptides in the pepmix that span the antigen overlap in sequence by 11 amino acids.

45. The method of any one of the preceding claims, wherein the patient is immunocompromised.

46. The method of any one of the preceding claims, wherein the patient is immunocompromised due to a treatment the patient received to treat a disease or condition.

47. The method of claim 46, wherein the treatment is a hematopoietic stem cell transplant, solid organ transplant, or anti-cancer agent.

48. The method of claim 46, wherein the treatment the patient received to treat a disease or condition is selected from the group consisting of reduced intensity conditioning, myeloablative conditioning, non-myeloablative conditioning, chemotherapy, and immunosuppressive drugs.

49. The method of claim 45, wherein the patient is immunocompromised due to age.

50. The method of claim 49, wherein the patent is less than 1 year of age.

51. The method of claim 49, wherein the patient is more than 65 years of age.

52. The method of claim 45, wherein the subject has an immune deficiency condition.

53. The method of claim 45, wherein the immune deficiency is primary immune deficiency.

54. The method of claim 45, wherein the subject has an HIV infection.

55. The method of any one of the preceding claims, wherein the patient is in need of a transplant therapy.

56. The method of claim 45, wherein the patient has a leukemia, myeloma, or lymphoma and is in need of a hematopoietic stem cell transplant therapy.

57. The method of any one of the preceding claims, wherein the first and/or one or more of each second T cell lines persist in vivo for at least 12 weeks.

58. The method of any one of the preceding claims, wherein the first and/or one or more of each second T cell lines persist in vivo for at least 12 weeks absent any active infection in the patient.
